(19) United States
(12) Patent Application Publication
(10) Pub. No.: US 2014/0018758 A1
(43) Pub. Date: Jan. 16, 2014

(54) STRETCHABLE LAMINATES FOR ABSORBENT ARTICLES AND METHODS FOR MAKING THE SAME

(71) Applicant: The Procter & Gamble Company, Cincinnati, OH (US)

(72) Inventors: Lalith JAYASINGHE, Liberty Township, OH (US); Audra MATHEWS, Cincinnati, OH (US); Jeanphilippe AUTRAN, Wyoming, OH (US); Antonius Lambertus De BEER, Loveland, OH (US); Brian UDENGAARD, Lystrup (DK)

(21) Appl. No.: 13/939,270

(22) Filed: Jul. 11, 2013

Related U.S. Application Data
(60) Provisional application No. 61/671,168, filed on Jul. 13, 2012.

(51) Int. Cl. A61F 13/514; A61F 13/538; A61F 13/513; A61F 13/49

(52) U.S. Cl. CPC A61F 13/51464; A61F 13/49009; A61F 13/538; A61F 13/513
USPC 604/370; 442/394; 442/398; 442/382

(57) ABSTRACT

A stretchable laminate for an absorbent article comprises a nonwoven substrate comprising a layer of spunbond fibers. A plurality of the spunbond fibers are formed from a composition comprising a thermoplastic polymer and an inorganic filler. The inorganic filler is present in the nonwoven substrate at a level of between about 1% and about 20% by weight of the nonwoven substrate. The stretchable laminate comprises an elastomeric material joined to a side of the nonwoven substrate.

### CROSS REFERENCE TO RELATED APPLICATION

This application claims the benefit of U.S. Provisional Patent Application Ser. No. 61/671,168, filed on Jul. 13, 2012, the entire disclosure of which is hereby incorporated by reference herein.

### FIELD

The present disclosure generally relates to stretchable laminates or laminates for absorbent articles, and, more particularly relates to stretchable laminates or laminates comprising a nonwoven substrate comprising a layer of spunbond fibers, wherein a plurality of the spunbond fibers comprise an inorganic filler.

### BACKGROUND

Stretchable laminates typically include a nonwoven substrate joined to an elastomeric material. These stretchable laminates are particularly useful when used to make at least one of the numerous elements that ultimately form disposable absorbent articles such as taped diapers, diaper pants, sanitary napkins, and/or adult incontinence products. For example, stretchable laminates may be used to make stretchable elements, such as stretchable ears, stretchable side panels, and/or stretchable outer covers for absorbent articles. Among other benefits, these stretchable laminates provide a better fit of the absorbent article on the wearer. A typical stretchable laminate that includes a nonwoven substrate bonded to an elastomeric material may be relatively hard to elongate by a caregiver or a user unless the laminate has first been mechanically “activated.” During mechanical activation, the stretchable laminate is strained to allow the laminate to at least partially recover some of the ease of elongation that the elastomeric material or film had before its bonding to the nonwoven substrate. Some nonwoven substrates, such as substrates made of at least a layer of carded fibers, are easily stretchable or elongatable even when bonded to an elastomeric material. During mechanical activation, a layer of carded fibers offers relatively little resistance and, as a result, a stretchable laminate that includes such a layer of carded fibers may be pre-strained to a great extent without causing either the layer of carded fibers or the elastomeric material to tear completely. The main drawback of using a layer of carded fibers is its cost in comparison to other nonwoven substrates, such as substrates that include a layer of spunbond fibers. The relatively inexpensive manufacturing process used to make spunbond type nonwoven substrates may make them particularly attractive for use in a stretchable laminate, but these nonwoven substrates are much more difficult to elongate without causing the spunbond layers and/or the elastomeric material to tear during the mechanical activation of the stretchable laminate. Due to their manufacturing process, spunbond layers may also have local variations in their basis weight that may cause the spunbond layers and the elastomeric material to tear during mechanical activation. A stretchable laminate whose elastomeric material is torn may not be able to be used and may need to be discarded causing undesirable waste and expenses. A stretchable laminate whose nonwoven substrate is repeatedly torn may be unpleasant to the touch when the laminate is elongated by a caregiver or a wearer. A nonwoven substrate that is partially or completely torn offers little or no resistance to limit the elongation of the overall stretchable laminate which in turn may potentially lead to the failure of the stretchable element made of the stretchable laminate if a caregiver or wearer elongates the stretchable elements abusively.

The present disclosure therefore provides stretchable laminates that comprise a nonwoven substrate comprising a layer of spunbond fibers joined to an elastomeric material to form a stretchable laminate that is able to better endure mechanical activation with reduced tearing or holes. The present disclosure also provides the stretchable laminates at a lower cost than conventional stretchable laminates. The present disclosure further provides an absorbent article having at least one element that includes such a stretchable laminate and methods for making the same.

### SUMMARY

In one form, the present disclosure is directed, in part, to a stretchable laminate or laminate for an absorbent article. The stretchable laminate comprises a nonwoven substrate comprising a layer of spunbond fibers. A plurality of, or all of, the spunbond fibers are formed from a composition comprising a thermoplastic polymer and an inorganic filler. The inorganic filler is present in the composition at a level of between about 5% and about 15% or between about 3% and about 20% by weight of the composition. The inorganic filler is present in the nonwoven substrate at a level of between about 5% to about 15% or between about 1% to about 20% by weight of the nonwoven substrate. The stretchable laminate comprises an elastomeric material joined to a side of the nonwoven substrate.

In another form, the present disclosure is directed, in part, to a stretchable laminate or laminate for an absorbent article. The stretchable laminate comprises a nonwoven substrate comprising a layer of spunbond fibers. A plurality of, or all of, the spunbond fibers are formed from a composition comprising a polyolefin and an alkaline carbonate. The alkaline carbonate is present in the composition at a level of between about 3% and about 20% or between about 5% and about 15% by weight of the composition. The inorganic filler is present in the nonwoven substrate at a level of between about 5% to about 15% or between about 3% to about 20% by weight of the nonwoven substrate. The stretchable laminate comprises an elastomeric material joined to a side of the nonwoven substrate.

In still another form, the present disclosure is directed, in part, to an absorbent article comprising a liquid pervious layer or topsheet, a liquid impervious layer or backsheet, and an absorbent core disposed between the liquid pervious layer and the liquid impervious layer. The absorbent article comprises a stretchable laminate or laminate joined to any of the liquid pervious layer, the liquid impervious layer, and the absorbent core. The stretchable laminate comprises a nonwoven substrate comprising a layer of spunbond fibers. A plurality of, or all of, the spunbond fibers are formed from a composition comprising a polyolefin and an inorganic filler. The inorganic filler is present in the composition at a level of between about 3% and about 20% or between about 5% and about 15% by weight of the composition. The inorganic filler is present in the nonwoven substrate at a level of between about 5% to about 15% or between about 3% to about 20% by weight of the nonwoven substrate. The stretchable laminate comprises an elastomeric material joined to a side of the nonwoven substrate.

In yet another form, the present disclosure is directed, in part, to a stretchable laminate or laminate for an absorbent article. The stretchable laminate comprises a nonwoven substrate comprising a layer of spunbond fibers. A plurality of, or all of, the spunbond fibers are formed from a composition comprising a thermoplastic polymer and an inorganic filler. The spunbond fibers each have a diameter. The inorganic filler in each of the fibers has an average or maximum particle size that is less than 90% of the diameter of the spunbond fiber. In an embodiment, the average particle size may be less than 90% of the diameter of the spunbond fiber and the maximum particle size may be larger than the diameter of the spunbond fiber. The stretchable laminate comprises an elastomeric material joined to a side of the nonwoven substrate.

In yet another form, the present disclosure is directed, in part, to a stretchable laminate or laminate for an absorbent article. The stretchable laminate comprises a nonwoven substrate comprising a layer of spunbond fibers. A plurality of the spunbond fibers are multi-component fibers comprising a core formed from a composition comprising a thermoplastic polymer and an inorganic filler present in the composition at a level of between about 5% and about 15% or between about 3% and about 20% by weight of the composition. The inorganic filler is present in the nonwoven substrate at a level of between about 5% to about 15% or between about 3% to about 20% by weight of the nonwoven substrate. The multi-component fibers further comprise a sheath comprising a thermoplastic polymer. The sheath may be free of the inorganic filler. The stretchable laminate comprises an elastomeric material joined to a side of the nonwoven substrate.

In yet another form, the present disclosure is directed, in part, to a method of making a stretchable laminate or laminate for an absorbent article. The method comprises providing a nonwoven substrate comprising a layer of spunbond fibers. A plurality of, or all of, the spunbond fibers are formed from a composition comprising a polyolefin and an inorganic filler. The inorganic filler is present in the composition at a level of between about 5% and about 15% or between about 3% and about 20% by weight of the composition. The inorganic filler is present in the nonwoven substrate at a level of between about 5% to about 15% or between about 3% to about 20% by weight of the nonwoven substrate. The method comprises providing an elastomeric material, and joining the nonwoven substrate to a side of the elastomeric material. In still another form, the present disclosure is directed, in part, to an absorbent article comprising a liquid pervious layer, a liquid impervious layer, an absorbent core disposed between the liquid pervious layer and the liquid impervious layer, and a side panel or ear joined to any of the liquid pervious layer, the liquid impervious layer, and the absorbent core. The side panel or ear comprises a first nonwoven substrate, a second nonwoven substrate, and an elastomeric material disposed intermediate the first nonwoven substrate and the second nonwoven substrate. The side panel or ear may comprise a fastening element. The first nonwoven substrate comprises a layer of spunbond fibers. A plurality of the spunbond fibers are formed from a composition comprising a polyolefin and an inorganic filler. The inorganic filler is present in the composition at a level of between about 5% and about 20% or between about 9% and about 13% by weight of the composition. The inorganic filler is present in the nonwoven substrate at a level of between about 5% to about 15% or between about 3% to about 20% by weight of the nonwoven substrate. The first nonwoven substrate further comprises a layer of meltblown fibers that are free of, or substantially free of, the inorganic filler. The second nonwoven substrate comprises a layer of spunbond fibers. A plurality of the spunbond fibers are formed from a composition comprising a polyolefin and an inorganic filler. The inorganic filler is present in the composition at a level of between about 3% and about 25% or between about 9% and 13% by weight of the composition. The inorganic filler is present in the nonwoven substrate at a level of between about 5% to about 15% or between about 3% to about 20% by weight of the nonwoven substrate. The second nonwoven substrate further comprises a layer of meltblown fibers. The meltblown fibers are free of, or substantially free of, the inorganic filler.

In still another form, the present disclosure is directed, in part, to an absorbent article comprising a liquid pervious layer, a liquid impervious layer, an absorbent core disposed between the liquid pervious layer and the liquid impervious layer, and a side panel or ear joined to any of the liquid pervious layer, the liquid impervious layer, and the absorbent core. The side panel or ear comprises a first nonwoven substrate, a second nonwoven substrate, and an elastomeric material disposed intermediate the first nonwoven substrate and the second nonwoven substrate. The side panel or ear may comprise a fastening element. The first nonwoven substrate comprises a layer of spunbond fibers. A plurality of the spunbond fibers are formed from a composition comprising a polyolefin and an inorganic filler. The inorganic filler is present in the composition at a level of between about 5% and about 20% or between about 9% and about 13% by weight of the composition. The inorganic filler is present in the nonwoven substrate at a level of between about 5% to about 15% or between about 3% to about 20% by weight of the nonwoven substrate. The first nonwoven substrate further comprises a layer of meltblown fibers that are free of, or substantially free of, the inorganic filler. The second nonwoven substrate comprises a layer of carded fibers.

### BRIEF DESCRIPTION OF THE DRAWINGS

The above-mentioned and other features and advantages of the present disclosure, and the manner of attaining them, will become more apparent and the disclosure itself will be better understood by reference to the following description of non-limiting embodiments of the disclosure taken in conjunction with the accompanying drawings, wherein:

FIG. 1 is a schematic cross-sectional view of a stretchable laminate in accordance with an embodiment of the present disclosure;

FIG. 2 is a schematic cross-sectional view of a stretchable laminate in accordance with another embodiment of the present disclosure;

FIG. 3 is a schematic cross-sectional view of a stretchable laminate in accordance with another embodiment of the present disclosure;

FIG. 4 is an SEM photograph of a portion of spunbond fibers formed from a composition comprising a thermoplastic polymer and about 5%, by weight, of an inorganic filler in accordance with an embodiment of the present disclosure;

FIG. 5 is an SEM photograph of a portion of spunbond fibers formed from a composition comprising a thermoplastic polymer and about 10%, by weight, of an inorganic filler in accordance with an embodiment of the present disclosure;

FIG. 6 is an SEM photograph of a portion of spunbond fibers formed from a composition comprising a thermoplastic polymer and about 15%, by weight, of an inorganic filler in accordance with an embodiment of the present disclosure;

FIG. 7 is an SEM photograph of a portion of spunbond fibers formed from a composition comprising a thermoplastic polymer and about 20%, by weight, of an inorganic filler in accordance with an embodiment of the present disclosure;

FIG. 8 is an SEM photograph of cut spunbond fibers formed from a composition comprising a thermoplastic polymer and about 15%, by weight, of an inorganic filler in accordance with an embodiment of the present disclosure;

FIG. 9 is an SEM photograph of a portion of a nonwoven substrate comprising a layer of spunbond fibers formed from a composition comprising a thermoplastic polymer and about 15%, by weight, of an inorganic filler in accordance with an embodiment of the present disclosure;

FIG. 10 is a schematic cross-sectional view of a bi-component fiber in accordance with an embodiment of the present disclosure;

FIG. 11 is a perspective view of a mechanical activation device for stretchable laminates that comprises two pressure applicators;

FIG. 12 is a cross-sectional view of teeth and recessed portions of the pressure applicators of the mechanical activation device of FIG. 11;

FIG. 13 is a schematic illustration of an absorbent article which may comprise the stretchable laminates and/or nonwoven substrates of the present disclosure;

FIG. 14 is a schematic illustration of a liquid impervious layer, an absorbent core, and a liquid pervious layer of an absorbent article;

FIG. 15 is a graph of Force per ligament per cm (N/cm) vs. Strain (%) of various nonwoven substrates of the present disclosure with the data obtained using a ring rolling simulation apparatus and method;

FIG. 16 is a graph of Force per ligament per cm (N/cm) vs. Strain (%) of various nonwoven substrates of the present disclosure with the data obtained using a ring rolling simulation apparatus and method; and

FIG. 17 is a plan view of a sanitary napkin which may comprise the stretchable laminates and/or nonwoven substrates of the present disclosure.

### DETAILED DESCRIPTION

Various non-limiting embodiments of the present disclosure will now be described to provide an overall understanding of the principles of the structure, function, manufacture, and use of the stretchable laminates for absorbent articles and methods for making the same disclosed herein. One or more examples of these non-limiting embodiments are illustrated in the accompanying drawings. Those of ordinary skill in the art will understand that the stretchable laminates for absorbent articles and methods for making the same described herein and illustrated in the accompanying drawings are non-limiting example embodiments and that the scope of the various non-limiting embodiments of the present disclosure are defined solely by the claims. The features illustrated or described in connection with one non-limiting embodiment may be combined with the features of other non-limiting embodiments. Such modifications and variations are intended to be included within the scope of the present disclosure.

### DEFINITIONS

As used herein, the term “absorbent article(s)” refers to consumer products whose primary function is to absorb and retain bodily exudates and wastes. Absorbent articles may refer to pants, taped diapers, adult incontinence products, and/or sanitary napkins (e.g., feminine hygiene products). The terms “diaper” and “pants” are used herein to refer to absorbent articles generally worn by infants, children, and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted, or otherwise disposed of in an environmentally compatible manner).

As used herein, the term “activated” refers to a material, such as a nonwoven substrate, laminate, or a stretchable laminate, which has been mechanically deformed or modified from its post-production state in order to increase the extensibility of (e.g., ring rolling) or otherwise modify of at least a portion of the material. A material may be activated by, for example, incrementally stretching the material in at least one direction. Other examples of such activation may be aperturing the material, creating structures in the material (e.g., soft tufts, scrubby tufts), changing the feel of the material (e.g., making it softer, rougher), and improving the fluid handling of the material by changing, for example, the permeability and/or the void volume.

As used herein, the term “carded fibers” refer to fibers that are of a discrete length which are sorted, separated, and at least partially aligned by a carding process. For example, a carded substrate refers to a substrate that is made from fibers which are sent through a combing or carding unit, which separates or breaks apart and aligns the fibers in, e.g., the machine direction to form a generally machine direction-oriented fibrous nonwoven web. Carded fibers may or may not be bonded after being carded.

As used herein, the term “film” refers generally to a relatively nonporous material made by a process that includes extrusion of, e.g., a polymeric material through a relatively narrow slot of a die. The film may be impervious to a liquid and pervious to an air vapor, but need not necessarily be so. Suitable examples of film materials are described in more detail hereinbelow.

As used herein, the term “joined” encompasses configurations wherein an element is directly secured to another element by affixing the element directly to the other element, and configurations wherein an element is indirectly secured to another element by affixing the element to intermediate member(s) which in turn are affixed to the other element.

As used herein, the term “laminate” refers to an element that has at least one nonwoven substrate joined to at least one elastomeric material or film. The laminate may have more than one nonwoven substrate joined to the at least one elastomeric material or film. The nonwoven substrates may be joined to the elastomeric material or film using bonding techniques or gluing techniques, for example.

As used herein, the term “layer” refers to a sub-component or element of a substrate. A “layer” may be in the form of a plurality of fibers made from a single beam on a multibeam nonwoven machine (for example a spunbond/meltblown/spunbond nonwoven substrate includes at least one layer of spunbond fibers, at least one layer of meltblown fibers, and at least one layer of spunbond fibers) or in the form of a film extruded or blown from a single die.

As used herein, the term “machine direction” or “MD” is the direction that is substantially parallel to the direction of travel of a substrate as it is made. Directions within 45 degrees of the MD are considered to be machine directional. The “cross direction” or “CD” is the direction substantially perpendicular to the MD and in the plane generally defined by the web. Directions within 45 degrees of the CD are considered to be cross directional.

As used herein, the term “meltblown fibers” refers to fibers made via a process whereby a molten material (typically a polymer), is extruded under pressure through orifices in a spinneret or die. High velocity hot air impinges upon and entrains the filaments as they exit the die to form filaments that are elongated and reduced in diameter and are fractured so that fibers of generally variable but mostly finite lengths are produced. This differs from a spunbond process whereby the continuity of the filaments is preserved along their length. An example meltblown process is found in U.S. Pat. No. 3,849,241 to Buntin et al.

As used herein, the term “nonwoven” refers to a porous, fibrous material made from continuous (long) filaments (fibers) and/or discontinuous (short) filaments (fibers) by processes such as, for example, spunbonding, meltblowing, carding, and the like. Nonwoven webs do not have a woven or knitted filament pattern.

As used herein, the term “pant” refers to disposable absorbent articles having a continuous perimeter waist opening and continuous perimeter leg openings designed for infant, child, or adult wearers. A pant may be configured with a continuous or closed waist opening and at least one continuous, closed, leg opening prior to the absorbent article being applied to the wearer. A pant may be preformed by various techniques including, but not limited to, joining together portions of the absorbent article using any refastenable and/or permanent closure member (e.g., seams, heat bonds, pressure welds, adhesives, cohesive bonds, mechanical fasteners, etc.). A pant may be preformed anywhere along the circumference of the absorbent article in the waist region (e.g., side fastened or seamed, front waist fastened or seamed, rear waist fastened or seamed). A pant may be opened about one or both of the side seams and then refastened. Example pants in various configurations are disclosed in U.S. Pat. Nos. 5,246,433, 5,569,234, 6,120,487, 6,120,489, 4,940,464, 5,092,861, 5,897,545, 5,957,908, and U.S. Patent Publication No. 2003/0233082.

As used herein, the term “spunbond fibers” refers to fibers made via a process involving extruding a molten thermoplastic material as filaments from a plurality of fine, typically circular, capillaries of a spinneret, with the filaments then being attenuated by applying a draw tension and drawn mechanically or pneumatically (e.g., mechanically wrapping the filaments around a draw roll or entraining the filaments in an air stream). The filaments may be quenched by an air stream prior to or while being drawn. The continuity of the filaments is typically preserved in a spunbond process. The filaments may be deposited on a collecting surface to form a web of randomly arranged substantially continuous filaments, which may thereafter be bonded together to form a coherent nonwoven layer. Example spunbond process and/or webs formed thereby may be found in U.S. Pat. Nos. 3,338,992, 3,692,613, 3,802,817, 4,405,297, and 5,665,300.

As used herein, the term “stretchable” refers to a material that, upon application of a biasing force, may stretch to an elongated length of at least 150% of its relaxed, original length (i.e., may stretch to 50% more than its original length), without complete rupture or breakage. In the event such a stretchable material recovers at least 40% of its elongation upon release of the applied force, the stretchable material will be considered to be “elastomeric.” For example, a stretchable material that has an initial length of 100 mm may extend at least to 150 mm, and upon removal of the force retracts to a length of at least 130 mm (i e, exhibiting a 40% recovery).

As used herein, the term “substrate” refers to an element that includes at least a fibrous layer and has enough integrity to be rolled, shipped and subsequently processed (for example a roll of a substrate may be unrolled, pulled, taught, folded and/or cut during the manufacturing process of an absorbent article having an element that includes a piece of the substrate). Multiple layers may be bonded together to form a substrate.

As used herein, the term “taped diaper” refers to disposable absorbent articles having an initial front waist region and an initial rear waist region that are not fastened, pre-fastened, or connected to each other as packaged, prior to being applied to the wearer. A taped diaper may be folded about its lateral central axis with the interior of one waist region in surface to surface contact with the interior of the opposing waist region without fastening or joining the waist regions together. Example taped diapers disclosed in various suitable configurations are illustrated in U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571.

While not intending to limit the utility of the stretchable laminates described herein, it is believed that a brief description of their characteristics as they may relate to the laminate manufacturing and intended use will help elucidate the stretchable laminates and methods of the present disclosure. In conventional stretchable laminates suitable for use, for example, as an element of an absorbent article, the laminates typically comprise at least a nonwoven substrate that is bonded to an elastomeric material or film. Modern absorbent articles such as taped diapers, pants, sanitary tissue products, and/or adult incontinence products include many elements that are at one time or another in contact with the caregiver or user's skin. The use of nonwoven substrates is particularly advantageous in such elements due to the soft feel and the cloth-like appearance they provide. Some modern disposable absorbent articles are also designed to provide an underwear-like fit. Some of the elements of modern absorbent articles are provided with elastomeric components which provide them with elastic properties and contribute not only to the performance, but also the underwear-like fit of these absorbent articles when worn. Non-limiting examples of such elements that include elastomeric components include ear panels of a diaper, side panels of a pant, or at least part of, if not all of, the outer cover, backsheet, or liquid impervious layer. Conventional stretchable laminates typically include at least a nonwoven substrate that is bonded to an elastomeric material or film. The laminate is then mechanically activated to at least partially recover some of the ease of elongation that the elastomeric material or film had prior to being bonded to the nonwoven substrate. Mechanical activation of the stretchable laminate is often achieved by passing at least a portion of the laminate between a pair of pressure applicators having three-dimensional surfaces which at least to a degree are complementary to one another as disclosed, for example, in U.S. Pat. No. 5,167,897 to Weber et al., issued Dec. 1, 1992, and assigned to The Procter and Gamble Company. Typical stretchable laminates include an elastomeric material or film and two separate nonwoven substrates that are respectively bonded on each side of the elastomeric material or film. Known nonwoven substrates that have been used to make stretchable laminates are nonwoven substrates made of carded fibers and nonwoven substrates that include one or more layers of spunbond fibers such as a spunbond/meltblown/spunbond substrate. During mechanical activation, a carded substrate offers relatively little resistance to its elongation and, as a result, a stretchable laminate that includes such a carded substrate may be pre-strained or activated to a great extent without causing either the carded substrate or the elastomeric material or film to tear completely. However, carded substrates can be rather costly in comparison to spunbond substrates. On the other hand, spunbond substrates tend to be much more difficult to elongate without causing the spunbond substrates and/or the elastomeric material or film to tear during the mechanical activation of the laminate. Since manufacturers of absorbent articles are under continuous pressure to reduce manufacturing cost and minimize manufacturing waste, it is believed that the stretchable laminates disclosed hereinafter may be a suitable alternative to conventional stretchable laminates. The foregoing considerations are addressed by the present disclosure, as will be clear from the detailed disclosure.

Stretchable laminates are at times difficult to stretch because of the fiber strength in the layers of fibers, such as in layers of spunbond fibers. Various additives or fillers have been added to a composition used to form the fibers (whether meltblown or spunbond) for various reasons. It has now been discovered that using layers of fibers formed from a composition comprising one or more thermoplastic polymers and one or more inorganic fillers or additives (the terms “filler” and “additive” are used interchangeably herein) in a stretchable laminate allows for better elongation of the nonwoven substrates without creating as many holes in the elastomeric material within the stretchable laminate during mechanical activation. As one can imagine, holes that appear in a stretchable laminate after elongation are not desirable. This is especially important when activated stretchable laminates comprising a nonwoven substrate and an elastomeric material or film are stretched in the cross-direction during donning of an absorbent article, for example. During the formation of a stretchable laminate, the nonwoven substrate should be structurally modified or mechanically activated without causing hole creation in the elastomeric material of the stretchable laminate. As such, the fibers of the nonwoven substrate should be extended or gently broken to prevent, or at least inhibit, the generation of excess energy being transferred to the elastomeric material which may cause the holes in the elastomeric material. Inclusion of the inorganic fillers into the nonwoven substrate lowers the degree of energy transfer to the elastomeric material, and, thereby reduces the risk of hole creation in the elastomeric material. Furthermore, using an inorganic filler in a nonwoven substrate may significantly reduce the production cost of the substrate in that the inorganic filler materials combined with the thermoplastic polymer are cheaper than carded fibers and pure spunbond fibers. The inorganic filler is cheaper than polypropylene. As such, mixing the inorganic filler with polypropylene before fiber formation will save cost.

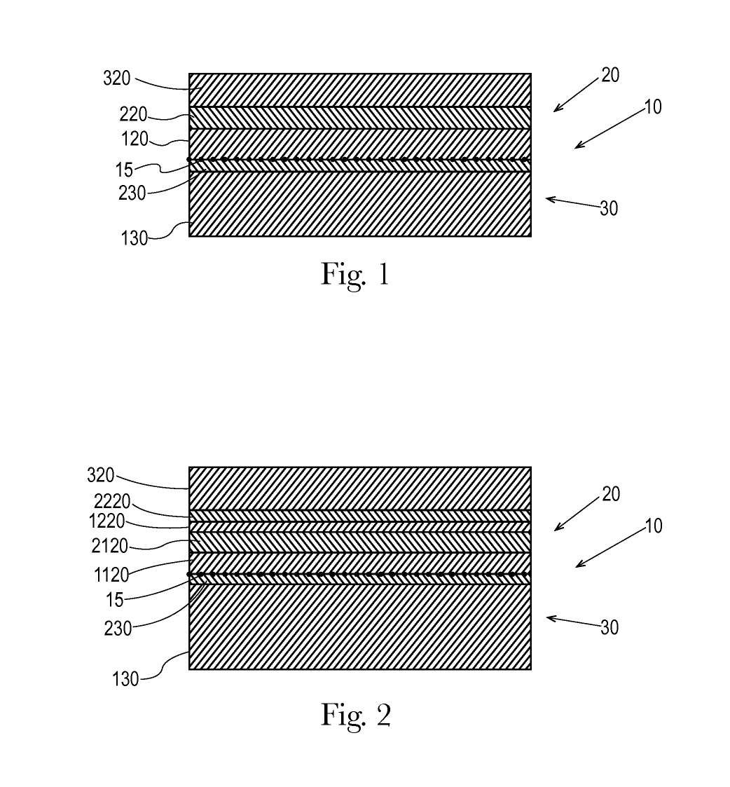

In an embodiment, referring to FIG. 1, a stretchable laminate 10 comprises a nonwoven substrate 20 that is bonded to an elastomeric material 30. The nonwoven substrate 20 comprises at least one layer 120 of spunbond fibers having top and bottom surfaces such that the bottom surface of the layer 120 is bonded to top surface or side of the elastomeric material 30 via an adhesive. The nonwoven substrate 20 may comprise additional layers such as for example at least one layer 220 of meltblown fibers (having top and bottom surfaces) and at least one layer 320 of spunbond fibers (also having top and bottom surfaces). The top surface of the layer 220 faces the bottom surface of the layer 320 and the top surface of the layer 120 faces the bottom surface of the layer 220.

The layer 120 of spunbond fibers may have a basis weight of between 2 g/m² (gsm) and 50 g/m², between 4 g/m² and 25 g/m², between 5 g/m² and 20 g/m², about 13 g/m², about 17 g/m², or about 20 g/m². The layer 220 of meltblown fibers may have a basis weight of between 0.5 g/m² and 10 g/m², between 0.5 g/m² and 8 g/m², between 1 g/m² and 5 g/m², about 13 g/m², about 17 g/m², or about 20 g/m². The layer 320 of spunbond fibers may have a basis weight of between 2 g/m² and 50 g/m², between 4 g/m² and 25 g/m², or even between 5 g/m² and 20 g/m², specifically reciting all 0.1 g/m² increments within the ranges specified in this paragraph. The basis weight of any of the substrates described herein may be determined using European Disposables and Nonwovens Association (“EDANA”) method 40.3-90. The basis weight of any of the individual layers described herein, and which together form a substrate, may be determined by running in sequence each of the fiber forming beams that are used to form separate layers and then measuring the basis weight of the consecutive formed layer(s) according to EDANA method 40.3-90. By way of example, the basis weight of each of the layers of an spunbond/meltblown/spunbond web (comprising a first layer of spunbond fibers, a layer of meltblown fibers, and a second layer of spunbond fibers) may be determined by first forming the first layer of spunbond fibers without forming the layer of meltblown fibers nor the second layer of spunbond fibers. The nonwoven that is produced includes only the first layer of spunbond fibers and its basis weight may be determined according to EDANA method 40.3-90. The basis weight of the layer of meltblown fibers may be determined by forming the first layer of spunbond fibers under the same conditions as in the previous step followed by formation of the layer of meltblown fibers laid on top of the first layer of spunbond fibers. The aggregate basis weight of the spunbond/meltblown web (which is again formed of the first layer of spunbond fibers and the layer of meltblown fibers) may be determined according to EDANA method 40.3-90. Since the basis weight of the first layer of spunbond fibers is known, the basis weight of the layer of meltblown fibers may be determined by subtracting the value of the basis weight of the first layer of spunbond fibers from the value of the aggregate basis weight of the spunbond/meltblown substrate. The basis weight of the second layer of spunbond fibers may be determined by forming the first layer of spunbond fibers and the layer of meltblown fibers under the same conditions as in the previous step followed by the formation of the second layer of spunbond fibers laid on top of the layer of meltblown fibers. The aggregate basis weight of the spunbond/meltblown/spunbond nonwoven may be determined according to EDANA method 40.3-90. Since the basis weight of the spunbond/meltblown web is known, the basis weight of the second layer of spunbond fibers may be determined by subtracting the value of the aggregate basis weight of the spunbond/meltblown web from the value of the aggregate basis weight of the spunbond/meltblown/spunbond web. The foregoing steps used to determine the basis weight of individual layers forming a substrate may be applied on as many layers as the ultimate nonwoven substrate includes. As previously discussed, the aggregate basis weight of the nonwoven substrate 20 is equal to the sum of the basis weight of each of its individual layers.

In an embodiment represented in FIG. 2, it may be advantageous to provide the nonwoven substrate 20 with at least two layers 1120, 2120 of spunbond fibers (each having top and bottom surfaces) in the portion of the substrate 20 that is disposed on the elastomeric material facing portion of the nonwoven substrate (i.e., the portion of the nonwoven substrate located between the layer 220 of meltblown fibers and the elastomeric material 30) instead of a single layer 120 of spunbond fibers. It is believed that the at least two separate layers of spunbond fibers may have a combined basis weight equal to the basis weight of the layer 120 of spunbond fibers and provide a greater level of performance than this single layer 120 during activation of at least a portion of the stretchable laminate. It is also believed that the at least two separate layers of spunbond fibers may have a combined basis weight that is less than the basis weight of a single layer 120 of spunbond fibers and provide the same level of performance as the single layer 120. By way of example, each of the layers of spunbond fibers 1120 and 2120 may have a basis weight of 6 g/m² as opposed to a single layer of spunbond fibers having a basis weight of at least 12 g/m². Each of the layers 1120 and 2120 of spunbond fibers may have a basis weight of between 1 g/m² and 25 g/m², between 2 g/m² and 12.5 g/m², or even between 2.5 g/m² and 10 g/m², specifically reciting all 0.1 g/m² increments within the above-specified ranges. It is believed that at least two separate layers of spunbond fibers provide greater basis weight homogeneity to the nonwoven substrate 20 and in particular to the elastomeric material facing portion of the nonwoven substrate 20. Without intending to be bound by any theory, it is also believed that since the elastomeric material facing portion of the nonwoven substrate 20 is the portion of the substrate that is directly bonded to the elastomeric material 30, a more homogeneous basis weight may help prevent local micro-tearing of the nonwoven substrate 20 during mechanical activation which may propagate to the elastomeric material 30 and cause the elastomeric material 30 to tear. It is believed that local micro-tearing of the nonwoven substrate 20 during mechanical activation may lead to an over-elongation of the portion of the elastomeric material 30 that is in the immediate vicinity of the micro-tear formed on the nonwoven substrate. This over-elongation of the elastomeric material 30 may result in the elastomeric material being torn or ruptured, in particular when the elastomeric material 30 is a film. It should be understood that the elastomeric material facing portion of the nonwoven substrate 20 may include more than two layers of spunbond fibers with an even lower basis weight to provide an even greater homogeneity.

In an embodiment, it may also be advantageous to provide the nonwoven substrate 20 with at least two layers 1220, 2220 of meltblown fibers (each having top and bottom surfaces) in the central portion of the substrate 20 instead of a single the layer 220 of meltblown fibers. The at least two separate layers 1220, 2220 of meltblown fibers may have a combined basis weight equal to the basis weight of the layer 220 of meltblown fibers and provide a greater level of performance than this single layer 220. In the alternative, the at least two separate layers of meltblown fibers may have a combined basis weight that is less than the basis weight of a single layer 220 of meltblown fibers and provide the same level of performance as the single layer 220. By way of example, each of the layers of meltblown fibers 1220 and 2220 may have a basis weight of 1 g/m² as opposed to a single layer of meltblown fibers having a basis weight of at least 2 g/m². Each of the layers 1220 and 2220 of meltblown fibers may have a basis weight of between 0.25 g/m² and 5 g/m², between 0.25 g/m² and 4 g/m², or even between 0.5 g/m² and 2.5 g/m², specifically reciting all 0.1 g/m² increments within the above-specified ranges. A layer 220 of meltblown fibers may be particularly advantageous when the layer 120 or layers 1120, 2120 of spunbond fibers disposed in the elastomeric material facing portion of the web 20 are adhesively bonded to the elastomeric material 30 with, for example, a hot melt adhesive (schematically represented by round dots 15 in FIGS. 1 and 2). It is believed that a meltblown layer 220 may prevent, or at least inhibit, the adhesive from reaching and even “bleeding though” the layer of spunbond fibers 320 which is the layer that may be in contact with the caregiver or wearer's skin. It is believed that two separate layers of meltblown fibers having a low basis weight are more effective at preventing adhesive “bleed-through” than a single layer of meltblown fibers having a higher basis weight. It is also believed that a layer 220 of meltblown fibers may conveniently be used as a “carrier layer” for additional smaller fibers such as nanofibers (i.e., fibers having a diameter of less than 1 μm). It is further believed that a layer 220 of meltblown fibers having a homogeneous basis weight may help achieve a more uniform coverage of any coating applied to the nonwoven substrate such as an adhesive coating, a printed ink, a surfactant, and/or a softening agent. It should be understood that the central portion (i.e., the portion of the substrate disposed between the outer layers of the substrate) of the nonwoven substrate 20 may include more than two layers 1220, 2220 of meltblown fibers with an even lower basis weight in order to provide an even greater homogeneity. One of ordinary skill will also appreciate that although the production of each of the layers 1120, 2120 of spunbond fibers and each or the layers 1220 and 2220 may require separate beams, it is believed that the production throughput of the nonwoven substrate may be increased. In the embodiment represented in FIG. 2, the top surface of the layer 1120 faces the bottom surface of the layer 2120, the top surface of the layer 2120 faces the bottom surface of the layer 1220, the top surface of the layer 1220 faces the bottom surface of the layer 2220, and the top surface of the layer 2220 faces the bottom surface of the layer 320.

In an embodiment, it may be also advantageous to provide the nonwoven substrate 20 with at least two layers of spunbond fibers in the portion of the substrate 20 that is facing away from the elastomeric material 30 (i.e., the portion of the nonwoven substrate positioned on top of the layer 220 of meltblown fibers) instead of a single layer 320 of spunbond fibers.

In one embodiment, the elastomeric material 30 may be an elastomeric nonwoven substrate or an elastomeric film. The elastomeric material 30 in the form of a film may include a core layer 130 that may be directly bonded to the spunbond layer 120 of the nonwoven substrate 20. A core layer 130 may be directly bonded to the nonwoven substrate 20 by extruding the elastomeric material 30 directly onto the nonwoven substrate 20. An adhesive may be added onto the contact surface of the extruded elastomeric material to increase the bond strength between the elastomeric material 30 and the nonwoven substrate 20. Non-limiting examples of suitable elastomeric materials include thermoplastic elastomers chosen from at least one of styrenic block copolymers, metallocene-catalyzed polyolefins, polyesters, polyurethanes, polyether amides, and combinations thereof. Suitable styrenic block copolymers may be diblock, triblock, tetrablock, or other multi-block copolymers having at least one styrenic block. Example styrenic block copolymers include styrene-butadiene-styrene, styrene-isoprene-styrene, styrene-ethylene/butylenes-styrene, styrene-ethylene/propylene-styrene, and the like. Commercially available styrenic block copolymers include KRATON® from the Shell Chemical Company of Houston, Tex.; SEPTON® from Kuraray America, Inc. of New York, N.Y.; and VECTOR® from Dexco Polymers, LP of Houston, Tex. Commercially available metallocene-catalyzed polyolefins include EXXPOL® and EXACT® from Exxon Chemical Company of Baytown, Tex.; AFFINITY®; and ENGAGE® from Dow Chemical Company of Midland, Mich. Commercially available polyurethanes include ESTANE® from Noveon, Inc., Cleveland, Ohio. Commercial available polyether amides include PEBAX® from Atofina Chemicals of Philadelphia, Pa. Commercially available polyesters include HYTREL® from E. I. DuPont de Nemours Co., of Wilmington, Del. Other particularly suitable examples of elastomeric materials include elastomeric polypropylenes. In these materials, propylene represents the majority component of the polymeric backbone, and as a result, any residual crystallinity possesses the characteristics of polypropylene crystals. Residual crystalline entities embedded in the propylene-based elastomeric molecular network may function as physical crosslinks, providing polymeric chain anchoring capabilities that improve the mechanical properties of the elastic network, such as high recovery, low set and low force relaxation. Suitable examples of elastomeric polypropylenes include an elastic random poly(propylene/olefin) copolymer, an isotactic polypropylene containing stereoerrors, an isotactic/atactic polypropylene block copolymer, an isotactic polypropylene/random poly(propylene/olefin) copolymer block copolymer, a reactor blend polypropylene, a very low density polypropylene (or, equivalently, ultra low density polypropylene), a metallocene polypropylene, and combinations thereof. Suitable polypropylene polymers including crystalline isotactic blocks and amorphous atactic blocks are described, for example, in U.S. Pat. Nos. 6,559,262, 6,518,378, and 6,169,151. Suitable isotactic polypropylene with stereoerrors along the polymer chain are described in U.S. Pat. No. 6,555,643 and EP 1 256 594 A1. Suitable examples include elastomeric random copolymers (RCPs) including propylene with a low level comonomer (e.g., ethylene or a higher α-olefin) incorporated into the backbone. Suitable elastomeric RCP materials are available under the names VISTAMAXX® (available from ExxonMobil, Houston, Tex.) and VERSIFY® (available from Dow Chemical, Midland, Mich.).

In some embodiments, the elastomeric materials may be coextruded with 3.5 μm/50 μm/3.5 μm skin/core/skin layer thicknesses. In some embodiments, the skin/core/skin layer thicknesses may be 3.5 μm/43 μm/3.5 μm, for example. In an embodiment, the skin layers may comprise 20% LDPE (Low-density polyethylene) having a density of 917 g/cm³ and a Melt Index in the range of about 2 to about 30 and 80% LLDPE (Linear low-density polyethylene) and have a density of 917 g/cm³ and a Melt Index in the range of about 2 to about 30, specifically reciting all 0.1 increments within the above-specified ranges. The skin layers may also comprise other compositions either in addition to or on lieu of the above. The core may comprise about 45% VECTOR® 8505 (SBS from Dexco Polymers, LP of Houston, Tex.), about 40% VECTOR® 7400 (oil extended SBS from Dexco Polymers, LP of Houston, Tex.), and about 15% Polystyrene (PS3190 from Nova Chemicals of Pittsburgh, Pa.). The core may also comprise other compositions in any suitable ranges either in addition to or on lieu of the above.

The elastomeric materials are not limited to any particular dimension, and may be configured as relatively thin sheets of material. In certain embodiments, the elastomeric materials (films) may have a thickness of between 1 μm-1 mm; 3 μm-500 μm; or 5 μm-100 μm, or any value within these ranges. Suitable basis weight ranges for the elastomeric films may include from 20 to 140 g/m², for example from 25 to 100 g/m²; from 30 to 70 g/m²; or even from 35 to 45 g/m², specifically including all 0.5 g/m² increments within the above-specified ranges. The elastomeric materials may be formed by any suitable methods known in the art such as, for example, extruding a molten thermoplastic and/or elastomeric polymer through a slit die and subsequently cooling the extruded sheet. Other non-limiting examples for making films include casting, blowing, solution casting, calendering, and formation from aqueous or cast, non-aqueous dispersions. Suitable methods of producing films from polymeric materials are described in Plastics Engineering Handbook of the Society of the Plastics Industry, Inc., Fourth Edition, 1976, pages 156, 174, 180 and 183).

It will be appreciated that materials that are typically used to form an elastomeric materials or films of the present disclosure may be tacky and cause the elastomeric material to stick to itself in the event the elastomeric material is rolled. It may be beneficial to provide at least one of the surfaces or sides of the core layer 130 with at least a skin layer 230 made of a material that does not stick to itself. Non-limiting examples of suitable materials for use as a skin layer include polyolefins such as polyethylene. Among other benefits, a skin layer 230 allows the elastomeric material 30 to be rolled for shipping and later unrolled for further processing. In one embodiment, the elastomeric material 30 or film may include a second skin layer disposed on the other surface or side of the core layer 130. The elastomeric material 30 or film may have a basis weight of between 10 g/m² and 150 g/m², between 15 g/m² and 100 g/m², or even between 20 g/m² and 70 g/m², specifically reciting all 0.1 g/m² increments within the above-specified ranges. The core layer 130 of the elastomeric material 30 may have a basis weight of between 10 g/m² and 150 g/m², between 15 g/m² and 100 g/m², or even between 20 g/m² and 70 g/m², and the skin layer 230 (if present) may have a basis weight of between 0.25 g/m² and 15 g/m², between 0.5 g/m² and 10 g/m², or even between 1 g/m² and 7 g/m², specifically reciting all 0.1 g/m² increments within the above-specified ranges.

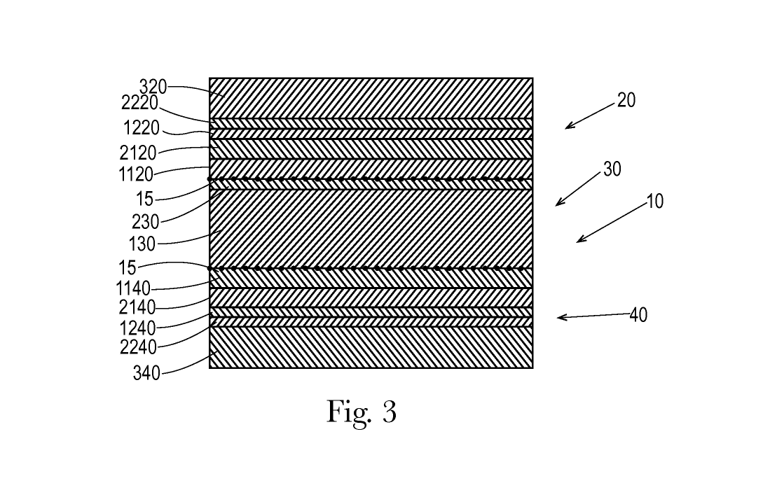

In an embodiment, referring to FIG. 3, the stretchable laminate 10 previously discussed in the context of FIG. 2 may additionally comprise a second nonwoven substrate 40 bonded to the other surface or side of the elastomeric material 30, such that the elastomeric material 30 is disposed between, or at least partially between, the nonwoven substrate 20 and the second nonwoven substrate 40. The second nonwoven substrate 40 may be a substrate of carded fibers or, in the alternative, a nonwoven substrate comprising at least one layer of spunbond and/or meltblown fibers. In an embodiment, the second nonwoven substrate 40 may comprise any of the layers previously discussed in the context of the nonwoven substrate 20 (i.e., nonwoven layers identified by reference numerals 120, 220, 320, 1120, 2120, 1220 and 2220). Consequently, the elastomeric material facing portion of the second nonwoven substrate 40 may include one (1140), two (1140, 2140) or more layers (1140, 2140, 340) of spunbond fibers. The central portion of the second nonwoven web 40 may include one (1240), two (1240, 2240) or more layers of meltblown fibers. In an embodiment, the nonwoven substrate 40 is bonded to the elastomeric material 30 such that it forms a mirror image, or a substantially mirror image, of the nonwoven substrate 20 relative to the elastomeric material 30. As such, it may be advantageous (although not required) for each of the nonwoven substrates 20 and 40 to be made of the same material and to include the same arrangement of layers in order to simplify the manufacturing process of the stretchable laminate 10.

In an embodiment, any of the previously discussed nonwoven layers 120, 320, 340, 1120, 1140, 2120, and 2140 of spunbond fibers may comprise or be formed from a composition comprising one or more thermoplastic polymers and one or more inorganic fillers. Likewise, any of the previously discussed nonwoven layers 220, 1220, 1240, 2220, and 2240 of meltblown fiber may comprise or be formed from a composition comprising one or more thermoplastic polymers and one or more inorganic fillers. In one embodiment, a nonwoven substrate may comprise spunbond fibers being formed from a composition comprising a thermoplastic polymer and an inorganic filler and meltblown fibers only comprising a thermoplastic polymer and being free of the inorganic filler. In various embodiments, the compositions may comprise one or more thermoplastic polymers, such as two types of polyolefins, for example.

Some thermoplastic polymers that may be used in the present disclosure are polyolefins, polyesters, polyamides, or halogen-containing polymers. These thermoplastic polymers may be used together or separately. The class of polyolefins includes, among others, polyethylene (HDPE, LDPE, LLDPE, VLDPE; ULDPE, UHMW-PE), polypropylene (PP), poly(1-butene), polyisobutylene, poly(1-pentene), poly(4-methylpent-1-ene), polybutadiene, polyisoprene, as well as different olefin copolymers. In addition to these, heterophase blends are also included in the polyolefins. For example, polyolefins, especially polypropylene or polyethylene, graft or copolymers made of polyolefins and α, β-unsaturated carboxylic acid or carboxylic acid anhydrides, polyesters, polycarbonate, polysulfone, polyphenylene sulfide, polystyrene, polyamides or a mixture of two or more of the mentioned compounds, may be used.

The polyesters include polyethylene terephthalate (PET), polytrimethylene terephthalate (PTT), polybutylene terephthalate (PBT), polyethylene aphthalate (PEN), but also degradable polyesters, like polylactic acid (polylactide, PLA).

The halogen-containing fiber-forming polymers include polyvinylchloride (PVC), polyvinylidene chloride (PVDC), polyvinylidene fluoride (PVDF) and polytetrafluoroethylene (PTFE).

In addition to the already mentioned fiber-forming synthetic polymers, there are other polymers, like polyacrylates, polyvinyl acetate, polyvinyl alcohol, polycarbonate, polyurethane, polystyrene, polyphenylene sulfide, polysulfone, polyoxymethylene, polyimide, or polyurea, for example, which may be considered as a component of the thermoplastic polymer fibers of the present disclosure.

The inorganic filler may be present in a composition of one or more thermoplastic polymers (before fiber formation through either meltblowing or spunbonding) at a level, by weight of the composition, of between about 1% and about 25%, of between about 3% and about 20%, of between about 5% and about 20%, of between about 5% and about 15%, of between about 8% and about 12%, about 5%, about 9%, about 10%, about 11%, about 11.5%, about 12%, about 13%, about 14%, and about 15%, specifically reciting all 0.1% increments within the above-specified ranges. As such, the composition used to form the fibers will have the above-recited weight percents of the inorganic filler with the balance being of one or more molten, melted, and/or flowable thermoplastic polymers. In some embodiments, the inorganic fillers may remain as solid particles when immersed in and mixed with the molten thermoplastic polymer owing to their higher melting temperature relative to the melting temperature of at least some thermoplastic polymers. In other embodiments, the inorganic filler materials may not remain as solid particles when immersed in and mixed with the molten thermoplastic polymer.

To create the composition used to form the fibers of the present disclosure, a batch of one or more molten thermoplastic polymers may be dosed with a mixture of inorganic filler/thermoplastic polymer or simply dosed directly with 100% inorganic filler. In an embodiment, the dosing mixture may have any suitable ratio, such as a ratio of 70% inorganic filler to 30% thermoplastic polymer. The 30% thermoplastic polymer may be molten, melted, and/or flowable to encapsulate or carry the 70% inorganic filler. Other ratios of inorganic filler to thermoplastic polymer may also be used for dosing the batch of the molten thermoplastic polymer and are within the scope of the present disclosure. The thermoplastic polymer in the 70%/30% inorganic filler/thermoplastic polymer mixture may be the same polymer as or a different polymer as the batch of the molten thermoplastic polymer to which the mixture is being added. In an embodiment, the mixture may be an about 70%/30% CaCO₃/polypropylene mixture and the molten thermoplastic polymer in the batch may be polypropylene. If, for example, it was desired to produce a 15% CaCO₃, by weight composition, a dosing of about 21.4% 70/30 CaCO₃/polypropylene mixture would be added to the batch of the molten polypropylene (i.e., 15%=0.7(X % (dosing percent)), thereby X %=21.4 dosing percent). To calculate the percent of CaCO₃ that will be in the composition and, thereby, the formed fibers, one would multiply the dosing percent by 0.7 (i.e., 21.4% (dosing percent)×0.7=15% in the composition). Of course, if a 60%/40% ratio of an inorganic filler/thermoplastic polymer mixture was used for dosing, 0.6 would be the number used in the equations above and so forth. Operators of nonwoven manufacturing lines may optimize particular settings of their processes to produce the spunbond or meltblown fibers and/or layers comprising inorganic fillers of the present disclosure.

Each of the fibers (e.g., meltblown or spunbond) of the nonwoven substrates of the present disclosure may have a diameter. The maximum size or average size of the particles of the inorganic filler in the fibers may be less than about 95% or 95%, less than about 90% or 90%, less than about 85% or 85%, less than about 80% or 80%, less than about 75% or 75%, less than about 60% or 60%, less than about 50% or 50%, less than about 40% or 40%, less than about 30% or 30%, less than about 25% or 25%, or less than about 20% or 20%, but greater than zero, of the diameter of the fibers. In other embodiments, the maximum size or average size of the particles of the inorganic filler is in the range of 1% to 99%, 1% to 90%, 5% to 90%, 1% to 60%, 10% to 90%, 20% to 95%, 20% to 90%, or 30% to 90%, or is about 65%, 60%, 55%, 50%, 45%, 35%, 30%, 25%, 20%, 15%, or 10% of the diameter of the fibers, specifically reciting all 0.1% increments within the above-specified ranges. In an embodiment, the maximum particle size of the inorganic fillers may be greater than the diameter of the fibers, such as 101%, 105%, 110%, 115%, 120%, 125%, 130%, or in the range of 101% to 200%, specifically reciting all 0.5% increments within the specified range, of the diameter of the fibers. In an embodiment, each particle of the inorganic filler in the formed fibers may have a maximum size or average size of about 0.1 microns to about 19 microns, about 0.5 microns to about 18 microns, about 1 micron to about 15 microns, less than about 18 microns, less than about 16 microns, less than about 15 microns, less than 14 about microns, about 0.5 microns to about 14 microns, about 2 microns to about 10 microns, about 3 microns to about 8 microns, and about 1.4 microns, specifically reciting all 0.1 micron increments within the above-specified ranges.

In an embodiment, a stretchable laminate for an absorbent article is provided. The stretchable laminate may comprise a nonwoven substrate comprising at least one layer of spunbond fibers and, optionally, at least one layer of meltblown fibers. A plurality of, or all of, the spunbond fibers may be formed from a composition comprising one or more thermoplastic polymers and one or more inorganic fillers. The spunbond fibers have a diameter and the one or more inorganic fillers in each of the fibers may have a maximum or average particle size (i.e., longest dimension) that is less than 90% (or other sizes as specified herein) of the diameter of the spunbond fibers. The stretchable laminate may comprise an elastomeric material joined to a side or first side of the nonwoven substrate. As an example embodiment, the maximum or average particle size of the inorganic filler may be in the range of about 1 micron to about 15 microns or about 3 microns to about 8 microns.

In an embodiment, the inorganic filler may comprise an alkaline carbonate, such as calcium carbonate, for example. It should be understood that calcium carbonate is normally obtained from natural chalk deposits, and that local geological conditions dictate the content of additional minerals in the chalk. Thus, metal oxides, like iron oxide, may also be contained in chalk, for example, in addition to other alkaline earth carbonates.

The use of different alkaline earth carbonates or a mixture of two or more of these compounds is naturally also conceivable. Calcium carbonate (CaCO₃), magnesium carbonate (MgCO₃), and/or barium carbonate (BaCO₃) are proposed, in particular. In some embodiments, the inorganic filler may comprise at least 90 wt %, 95 wt %, or 97 wt % calcium carbonate.

Additional fillers, one or more of which are usable with or without an alkaline earth carbonate, include iron oxides, aluminum oxide (Al₂O₃), finely powered silica, silicon dioxide (SiO₂), calcium oxide (CaO), magnesium oxide (MgO), barium sulfate (BaSO₄), magnesium sulfate (MgSO₄), aluminum sulfates (AlSO₄), or aluminum hydroxide (AlOH₃). Clays (kaolin), zeolites, kieselguhr, talc, mica, or carbon black are also considered. Titanium dioxide (TiO₂) may also be used as an inorganic filler. However, at higher calcium carbonate contents, the addition of the matting agent titanium dioxide (TiO₂) may be fully dispensed with. This circumstance is worth noting with respect to the present disclosure, because titanium dioxide is more expensive than calcium carbonate and an additional cost advantage is therefore gained.

In some embodiments, the inorganic fillers may be alkaline halides, such as calcium chloride and magnesium chloride, alkaline oxides, such as calcium oxide and magnesium oxide, and alkaline sulfates, such as calcium sulfate and magnesium sulfate. In other embodiments, the inorganic fillers may be alkali carbonates, such as sodium carbonate and potassium carbonate, alkali halides, such as sodium chloride, sodium bromide, and potassium chloride) and alkali sulfates, such as sodium sulfate and potassium sulfate. Any combination of the inorganic fillers described herein may also be used. In still other embodiments, any other suitable filler materials, inorganic or organic, may be used with the present disclosure.

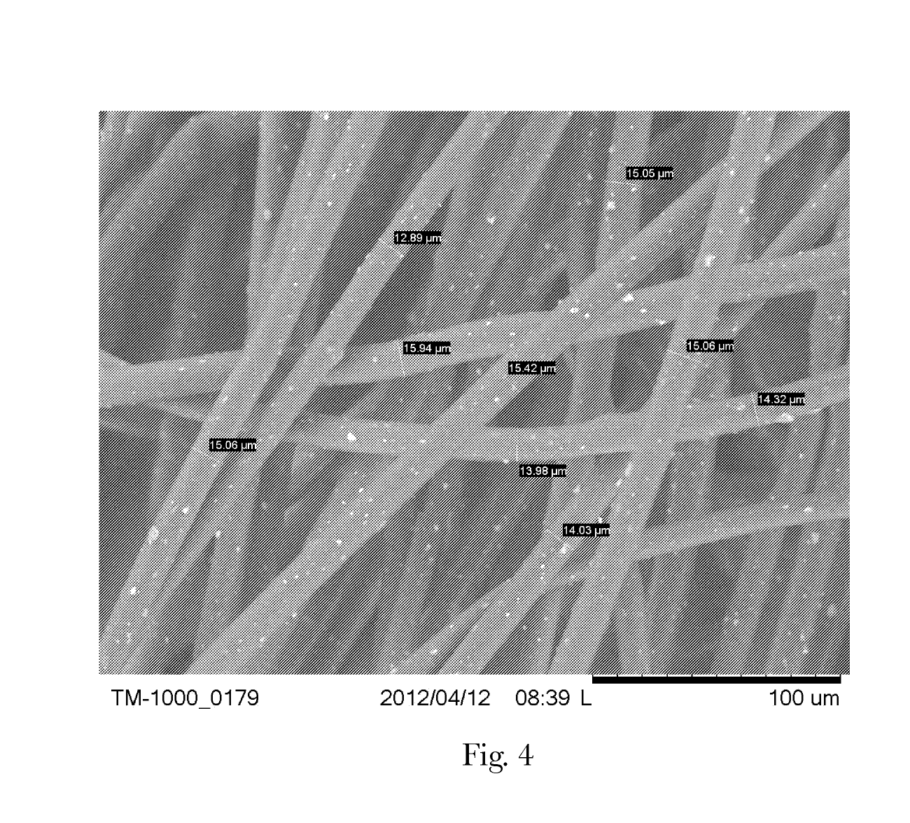

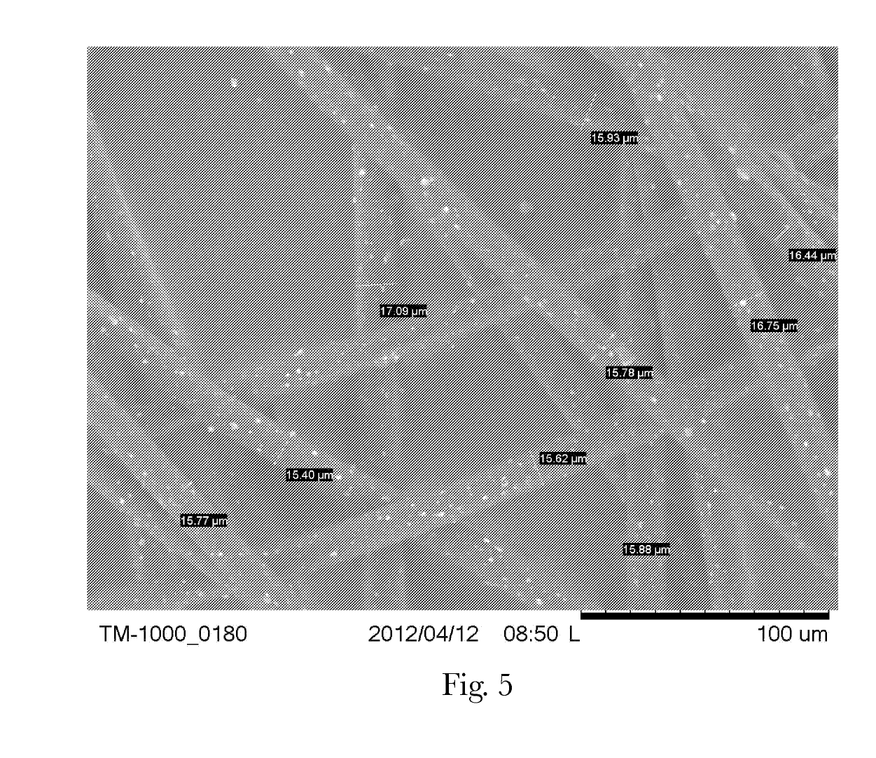

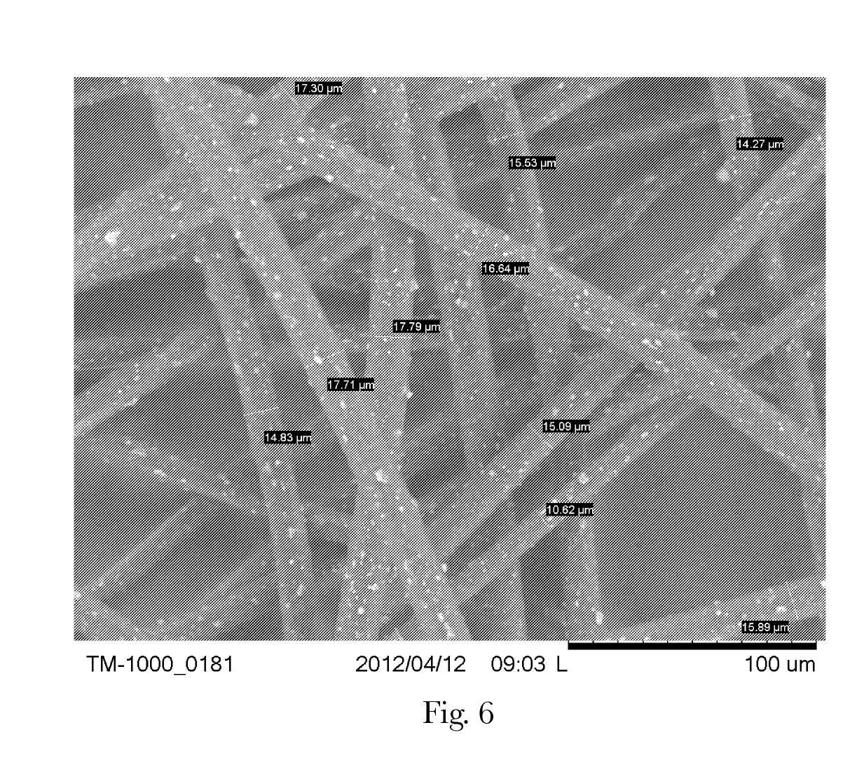

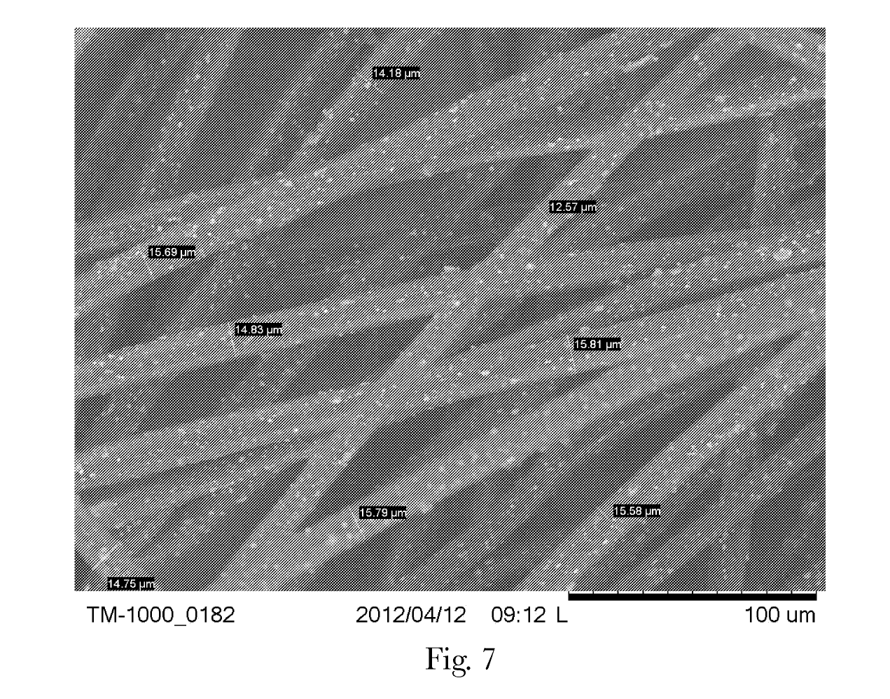

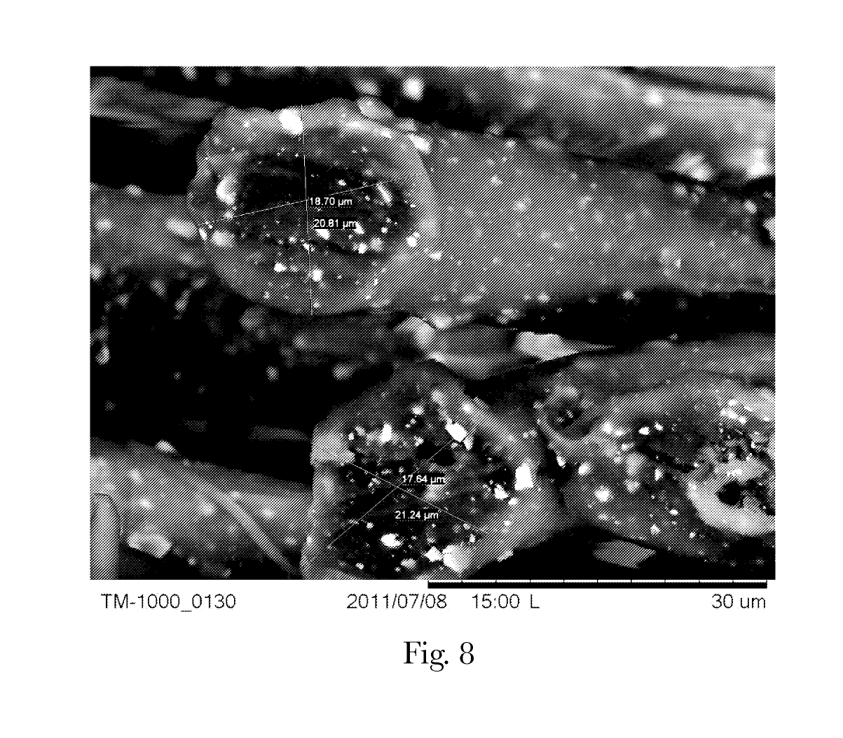

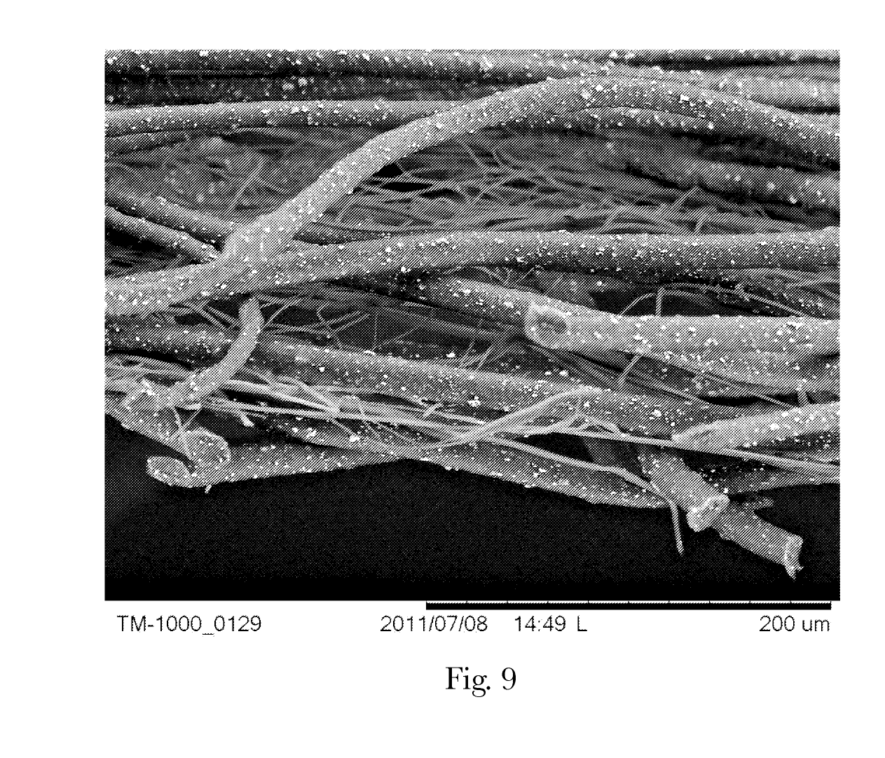

FIG. 4 is an SEM (Scanning Electron Microscope) photograph of a portion of a nonwoven substrate, wherein fibers of a spunbond layer of the nonwoven substrate are formed from a composition comprising polypropylene and about 5% calcium carbonate, by weight of the composition. FIG. 5 is an SEM photograph of a portion of a nonwoven substrate, wherein fibers of a spunbond layer of the nonwoven substrate are formed from a composition comprising polypropylene and about 10% calcium carbonate, by weight of the composition. FIG. 6 is an SEM photograph of a portion of a nonwoven substrate, wherein fibers of a spunbond layer of the nonwoven substrate are formed from a composition comprising polypropylene and about 15% calcium carbonate, by weight of the composition. FIG. 7 is an SEM photograph of a portion of a nonwoven substrate, wherein fibers of a spunbond layer of the nonwoven substrate are formed from a composition comprising polypropylene and about 20% calcium carbonate, by weight of the composition. FIG. 8 is an SEM photograph of cross-sectional views of two fibers of a spunbond layer of a nonwoven substrate that is about 13 gsm, wherein the fibers are formed from a composition comprising polypropylene and about 11.5% calcium carbonate, by weight of the composition. FIG. 9 is an SEM photograph of a portion of a nonwoven substrate that is about 13 gsm, wherein fibers of spunbond layers of the nonwoven substrate are formed from a composition comprising polypropylene and about 11.5% calcium carbonate, by weight of the composition.

In an embodiment, the inorganic filler, such as calcium carbonate, may be coated with, for example, at least one organic material. The at least one organic material may be chosen from fatty acids, including but not limited to stearic acid, and salts and esters thereof, such as stearate.

In another embodiment, the at least one organic material may be ammonium stearate. In another embodiment, the at least one organic material may be calcium stearate. In yet a further embodiment, the at least one organic material may be salts and esters of fatty acids. The product FiberLink™ 101 S sold by Imerys, Inc. is a non-limiting example of a calcium carbonate product coated with stearic acid. Surface coating the inorganic filler with at least one organic material may serve to improve dispersion of the filler particles throughout the fiber and facilitate the overall production of the fibers.

Coated calcium carbonate products suitable for use in the fibers of the present disclosure include, but are not limited to, those commercially available. In one embodiment, the coated calcium carbonate is chosen from those products sold under the names FiberLink™ 101S and 103S by Imerys, Inc. In another embodiment, the coated calcium carbonate is the product sold under the name MAGNUM GLOSS® by the Mississippi Lime Company. In a further embodiment, the coated calcium carbonate is the product sold under the name ALBAGLOS® by Specialty Minerals, Inc. In yet another embodiment, the coated calcium carbonate is the product sold under the name OMYACARB® by OMYA, Inc. In yet a further embodiment, the coated calcium carbonate is the product sold under the name HUBERCARB® by Huber, Inc. In still another embodiment, the coated calcium carbonate is the product sold under the name SUPERCOAT® by Imerys, Inc. The commercially available coated calcium carbonate products may be available in the form of dry powders having defined particle size ranges; however, all commercial coated calcium carbonate products may not exhibit a particle size and distribution appropriate for use in accordance with the present disclosure. The particle size of the inorganic filler may affect the maximum amount of the filler that can be effectively incorporated into the nonwoven fibers disclosed herein, as well as the aesthetic properties and strength of the resulting products. In other embodiments, the inorganic fillers may not be coated with any materials or may be coated with other inorganic materials.

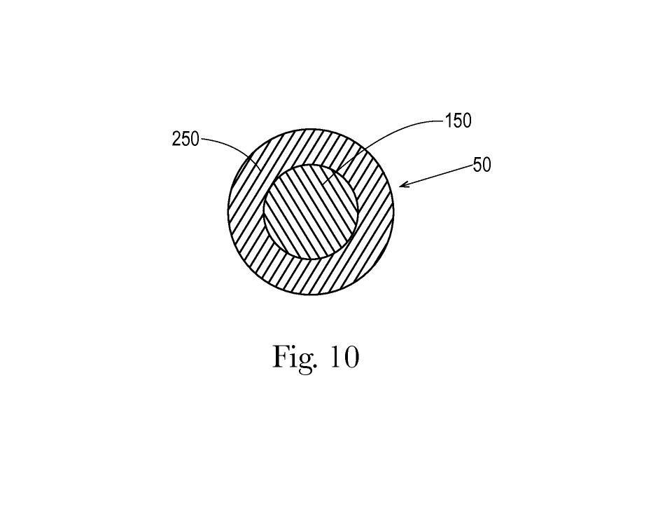

In an embodiment, any of the previously discussed nonwoven layers of spunbond fibers or meltblown fibers may comprise or be made of bi-component or multi-component fibers comprising one or more thermoplastic polymers and one or more inorganic fillers. In some embodiments, only the spunbond fibers may comprise or be made of bi-component or multi-component fibers. Referring to FIG. 10, the fibers 50 may each comprise a core 150 and a sheath 250. The core 150 may be formed from a composition comprising one or more thermoplastic polymers and one or more inorganic fillers. In an embodiment, the thermoplastic polymer may comprise or be a polyolefin, such as polypropylene, and the inorganic filler may comprise or be calcium carbonate. The core 150 may also be formed from a composition comprising another thermoplastic polymer and another inorganic filler as discussed herein. The sheath 250 may comprise one or more thermoplastic polymers, such as a polyolefin, and may have little (e.g., less than 10%, less than 5%, less than 1%, or less than 0.5%) or no inorganic filler. The polyolefin used to make the core 150 may be different than or the same as the polyolefin used to make the sheath 250. If two polyolefins are used, both polyolefins may have different melt temperatures and different tensile properties. In an embodiment, each of the two polyolefin polymers used to form the bi-component fibers may be substantially non-elastic. The two polyolefins may be polypropylene and polyethylene, for example. In an embodiment, the sheath 250 sheath may contain less than 1% of an inorganic filler, a thermoplastic polymer of the sheath may comprise polyethylene, and a thermoplastic polymer of the core may comprise polypropylene. Bi-component fibers may have any configuration known in the art, but it is believed that the bi-component fibers 50 as represented in FIG. 10 having the core 150 distinct from the sheath 250 may be advantageous in particular when the core 150 comprises a first polymer having a first melt temperature and the sheath 250 comprises a second polymer having a second melt temperature that is lower than melt temperature of the first polymer. In one embodiment, the melt temperature of the first polymer forming the core may be at least 130° C., at least 140° C., or even at least 150° C. The melt temperature of the second polymer forming the sheath may be less than 150° C., less than 140° C., or even less than 130° C. The melt temperature of a polymer may be determined according to ASTM D 3418. In one embodiment, the first polymer forming the core 150 may have a density of at least 0.9 g/cc, at least 0.92 g/cc, or at least 0.95 g/cc. The second polymer forming the sheath 250 may have a density of less than 0.95 g/cc, less than 0.92 g/cc, or less than 0.9 g/cc. The density of a polymer may be determined according to ASTM D 792. Bi-component or multi-component fibers with a core comprising the inorganic filler and a sheath free of, or substantially free of, the inorganic filler, may be advantageous in preventing, or at least inhibiting, the inorganic filler from degrading absorbent article manufacturing equipment during mechanical activation or other processing steps. It may also reduce absorbent article manufacturing equipment contamination from the inorganic fillers. In an embodiment, a nonwoven substrate in a stretchable laminate may comprise two or more spunbond layers therein. The spunbond layers that will contact the absorbent article manufacturing equipment may contain little or no inorganic material, while other spunbond layers either in the center of the nonwoven substrate or adjacent to the elastomeric material may comprise between 3% and 25% by weight of the inorganic filler. This type of layering of the spunbond layers within the stretchable laminates may help reduce wear on absorbent article manufacturing equipment and absorbent article manufacturing equipment contamination both potentially caused by the inorganic fillers since the spunbond layers contacting the absorbent article manufacturing equipment will have little or no inorganic fillers. Such positioning of the spunbond layers within stretchable laminates and/or nonwoven substrates also applies in the same way to the mono-component fibers (i.e., thermoplastic polymer and inorganic filler without a core/sheath) discussed herein. In an embodiment of a bi-component fiber, the core may comprise a first amount, by weight, of the inorganic filler and the sheath may comprise a second amount, by weight, of the inorganic filler. The first amount may be the same as or different than the second amount. Further details regarding bi-component or multi-component fibers and methods of making the same may be found in U.S. Patent Application Publ. Nos. 2009/0104831, 2010/0262107, 2010/0262105, 2010/0262102, and 2010/0262103.

In an embodiment, a bi-component or multi-component fiber may comprise a core formed from a composition comprising one or more elastomeric thermoplastic polymers, such as an elastomeric thermoplastic starch, a resin (e.g., VISTAMAXX® (available from ExxonMobil, Houston, Tex.), and/or a polylactic acid, for example. The elastomeric thermoplastic polymers, when used to form a core, may provide better stretch properties than typical thermoplastic polymers. Suitable elastomeric thermoplastic polymers are disclosed in U.S. Pat. No. 7,491,770 to Autran et al. Other suitable elastomeric thermoplastic polymers are known to those of skill in the art. The composition may also comprise polypropylene and an inorganic filler (e.g., an alkaline carbonate) in addition to the elastomeric thermoplastic polymer(s). The sheath of the fiber may be formed from a composition comprising one or more thermoplastic polymers and one or more inorganic fillers. The inorganic filler(s) may be present in the composition at a level between 5% and 15% or 5% to 20% by weight of the composition, specifically reciting all 0.5% increments within the above-specified ranges. In an embodiment, the thermoplastic polymer may be polypropylene and the inorganic filler may be an alkaline carbonate. The fibers of the present disclosure (e.g., spunbond fibers) may comprise these bi-component or multi-component fibers.

Fibers made from a composition comprising an inorganic filler and a thermoplastic polymer have a greater density than the density of fibers made from a composition comprising only a thermoplastic polymer. This is due to the fact that the inorganic filler has a density that is greater than the density of a thermoplastic polymer. In an embodiment, the inorganic filler comprises or is calcium carbonate and the thermoplastic polymer comprises or is polypropylene. In such an embodiment, the density of the calcium carbonate is greater than the density of the polypropylene. Owing to the fact that fibers comprising inorganic fillers are denser than fibers not comprising inorganic fillers and only comprising thermoplastic polymers, less fibers will be present in nonwoven substrates formed from such fibers when compared to nonwoven substrates formed from fibers only comprising a thermoplastic polymer, when the two nonwoven substrates have the same basis weight.

In various nonwoven substrates of the present disclosure, one or more of the spunbond layers may comprise fibers comprising one or more inorganic fillers. In an embodiment, no meltblown layers may be present in a nonwoven substrate, e.g., an SSS nonwoven substrate. In some embodiments, meltblown layers of nonwoven substrates may comprise fibers not comprising any inorganic fillers or comprising a small amount of inorganic fillers (e.g., less than 10%, less than 5%, or less than 1%). In an embodiment, one or more meltblown layers of a nonwoven substrate may comprise fibers comprising less inorganic filler than one or more spunbond layers in the same nonwoven substrate. If the meltblown fibers do comprise inorganic fillers, the average particle size and/or maximum particle size of the inorganic fillers within the meltblown fibers may be smaller than the average particle size and/or maximum particle size of the inorganic fillers present in any of the spunbond fibers owing to the smaller size of the meltblown fibers when compared to the spunbond fibers. In other embodiments, the particle size range of the inorganic filler in the spunbond fibers may be larger, smaller, or different than the particle size range of the inorganic filler in the meltblown fibers. The inorganic filler in the spunbond fibers may be the same or different than the inorganic filler in the meltblown fibers. Likewise, the thermoplastic polymer in the spunbond fibers may be the same as or different than the thermoplastic polymer in the meltblown fibers. The nonwoven substrates may comprise a layer of carded fibers or be free from any carded fibers. In an embodiment, spunbond fibers in a nonwoven substrate may have a first amount, by weight, of one or more inorganic fillers, such as calcium carbonate, and meltblown fibers in the nonwoven substrate may have a second amount, by weight of one or more inorganic fillers. The first amount may be the same as, different than, greater than, or less than the second amount. The one or more inorganic fillers may be the same or different. In an embodiment of a nonwoven substrate, one of or both of the spunbond fibers and the meltblown fibers may comprise bi-component fibers comprising a core and a sheath. In some other embodiments of nonwoven substrates, different layers of spunbond fibers may comprise different inorganic fillers or different amounts, by weight, of inorganic fillers.

In an embodiment, the nonwoven substrates of the present disclosure may be used separate from the elastomeric materials. Some applications may be a topsheet or other portion of a sanitary napkin or an apertured topsheet or other portion of an absorbent article, such as a diaper. The nonwoven substrates may also be used in other product applications. The nonwoven substrates may be mechanically activated or SELFed. The terms “SELF” refers to Procter & Gamble Company technology in which SELF stands for Structural Elastic Like Film. Processes, apparatus, and patterns produced via SELF are illustrated and described in U.S. Pat. Nos. 5,518,801, 5,691,035, 5,723,087, 5,891,544, 5,916,663, 6,027,483, and 7,527,615 B2. While the process was originally developed using tooth geometries that would deform a polymer film without producing apertures, other tooth geometries have been developed that are more conducive to forming tufts (in the case of a nonwoven substrate) or tents (in the case of a film) with apertures on the leading and trailing ends. A process using SELF'ing to form tufts with apertures in a nonwoven web is disclosed in U.S. Pat. No. 7,682,686 B2.

In an embodiment, the density of the thermoplastic polymers may be less than the density of the inorganic material. The density of the thermoplastic polymers may be between 1% and 99% or 20% to 90% less than the density of the inorganic material, specifically reciting all 0.1% increments with the above-referenced range. The density of the thermoplastic polymers may be 95% less, 90% less, 85% less, 80% less, 75% less, 70% less, 65% less, 60% less, 55% less, 50% less, 45% less, 40% less, 37% less, 36% less, 35% less, 30% less, 25% less, 20% less, 15% less, 10% less, 5% less, 3% less, or 1% less, than the density of the inorganic materials.

In an embodiment, the density of fibers formed from a composition having only one or more thermoplastic polymers may be less than the density of fibers formed from a composition having one or more thermoplastic polymers and one or more inorganic materials. The density of fibers having only one or more thermoplastic polymers may be between 1% and 99% or 20% to 90% less than the density of fibers having one or more thermoplastic polymers and one or more inorganic materials, specifically reciting all 0.1% increments with the above-referenced range.

The density of fibers having only one or more thermoplastic polymers may be 95% less, 90% less, 85% less, 80% less, 75% less, 70% less, 65% less, 60% less, 55% less, 50% less, 45% less, 40% less, 37% less, 36% less, 35% less, 30% less, 25% less, 20% less, 15% less, 10% less, 5% less, 3% less, or 1% less, than the density of fibers having one or more thermoplastic polymers and one or more inorganic materials.

In an embodiment, the density of the polypropylene is about 0.91 g/m³ and the density of the uncoated calcium carbonate is about 2.71 g/m³.

Mechanical Activation of a Stretchable Laminate:

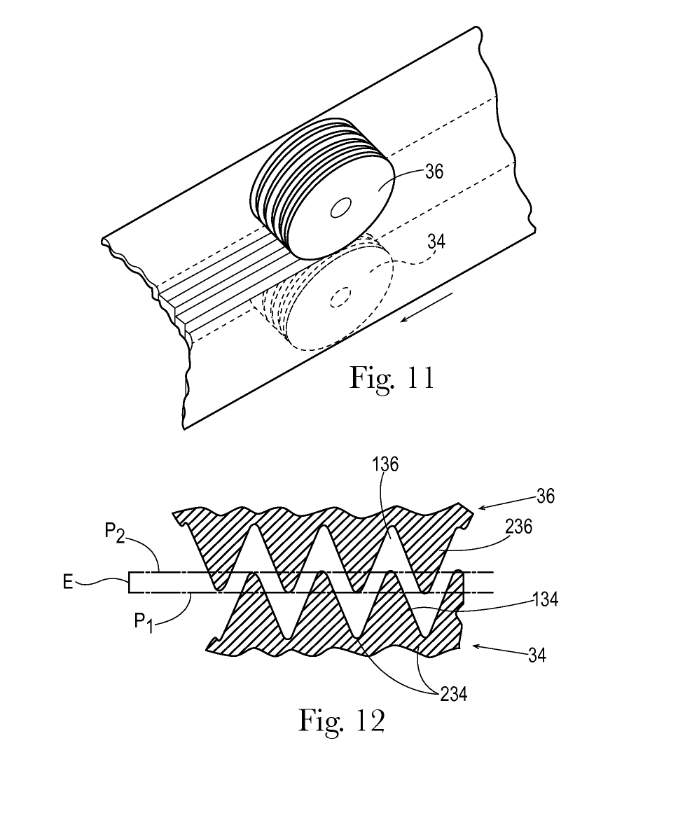

Any of the previously disclosed stretchable laminates may be mechanically activated (i.e., pre-strained) such that the stretchable laminate recovers some of the elasticity it lost when all the nonwoven substrates or elastomeric materials forming the stretchable laminate are bonded together. A non-limiting example of a process for mechanically activating a stretchable laminate is schematically represented in FIGS. 11 and 12. The device shown in those figures includes a pair of pressure applicators 34, 36 having three-dimensional surfaces, which at least to a degree, are complementary to one another. A pressure applicator (or roller) includes at least one engaging portion or tooth 134 (but may also include a plurality) corresponding to a recessed portion 136 of the other pressure applicator. A pressure applicator may include a plurality of engaging portions or teeth 134 and recessed portions 234 that may intermesh with a corresponding recessed portions 136 and engaging portions or teeth 236 on the other pressure applicator. When the stretchable laminate passes in between the pressure applicators 34, 36, portions of the stretchable laminate are strained. The stretchable laminate is able to relax and return substantially to its original width as it “exits” the pressure applicators. The degree of mechanical activation may be adjusted by varying the number of engaging portions and recessed portions and the depth of engagement of the pressure applicators on the stretchable laminate. One of ordinary skill in the art will appreciate that other processes for mechanically activating a stretchable laminate may be used and still provide the same benefits.

With reference to FIG. 12, which shows a portion of the intermeshing of the engaging portions 134 and 236 of pressure applicators 34 and 36, respectively, the term “pitch” refers to the distance between the apexes of adjacent engaging portions. The pitch may be between about 0.02 to about 0.30 inches (0.51-7.62 mm), or is between about 0.05 and about 0.15 inches (1.27-3.81 mm), specifically reciting all 0.01 increments within the specified range. The height (or depth) of the teeth is measured from the base of the tooth to the apex of the tooth, and may be equal for all teeth. The height of the teeth may be between about 0.10 inches (2.54 mm) and 0.90 inches (22.9 mm), specifically reciting all 0.05 inch increments within the specified range, and may be about 0.25 inches (6.35 mm) and 0.50 inches (12.7 mm) The engaging portions 134 in one pressure applicator may be offset by one-half the pitch from the engaging portions 236 in the other pressure applicator, such that the engaging portions of one pressure applicator (e.g., engaging portion 134) mesh in the recessed portions 136 (or valleys) located between engaging portions in the corresponding pressure applicator. The offset permits intermeshing of the two pressure applicators when the pressure applicators are “engaged” or in an intermeshing, operative position relative to one another. In an embodiment, the engaging portions of the respective pressure applicators are only partially intermeshing. The degree to which the engaging portions on the opposing pressure applicators intermesh is referred to herein as the “depth of engagement” or “DOE” of the engaging portions. As shown in FIG. 12, the DOE is the distance between a position designated by plane P1, where the apexes of the engaging portions on the respective pressure applicators are in the same plane (0% engagement), to a position designated by plane P2, where the apexes of the engaging portions of one pressure applicators extend inward beyond the plane P1 toward the recessed portions on the opposing pressure applicator. The optimum or effective DOE for particular laminates is dependent upon the height and the pitch of the engaging portions and the materials of the stretchable laminate. In other embodiments, the teeth of the mating rolls need not be aligned with the valleys of the opposing rolls. That is, the teeth may be out of phase with the valleys to some degree, ranging from slightly offset to greatly offset.

The stretchable laminates may be mechanically activated using a first DOE on a first set of pressure applicators and then mechanically activated again using a second DOE on a second set of pressure applicators. The second DOE may be the same as or greater than the first DOE. The second DOE mechanical activation may occur downstream of the first DOE mechanical activation. Stated another way, the mechanical activation using the first DOE stretches the stretchable laminate to a first extent and the mechanical activation using the second DOE stretches the stretchable laminate to a second extent, wherein the first extent is less than or the same as the second extent. The DOE is generally discussed in mm and, in an embodiment, may be in the range of about 4 mm to about 7 mm, for example.

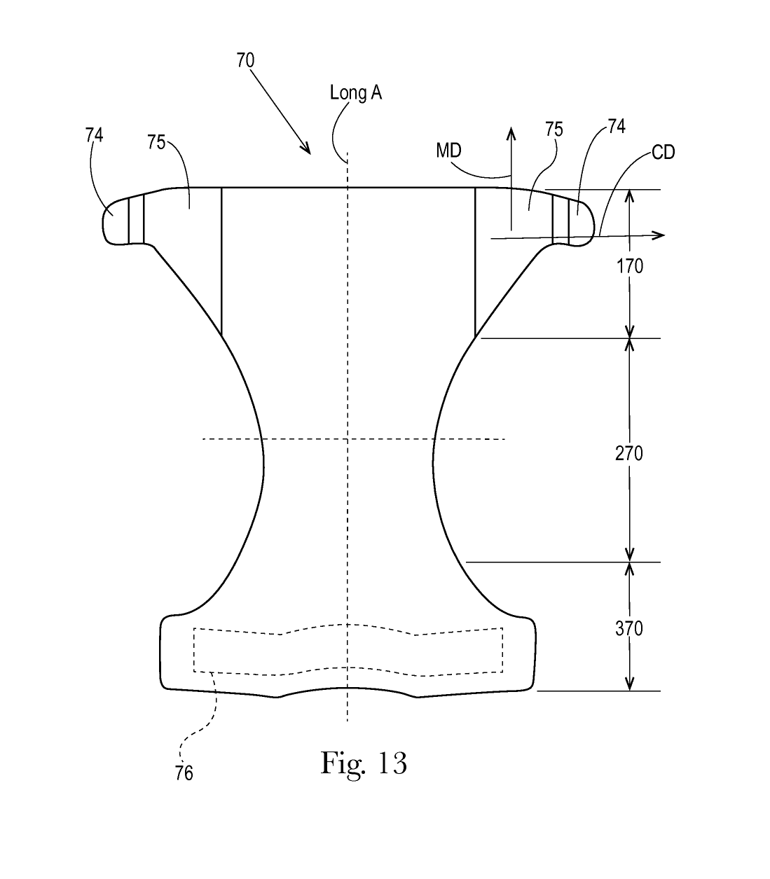

A stretchable laminate comprising any of the nonwoven substrates or elastomeric materials discussed herein may be adapted for use in a disposable absorbent article such as a taped diaper, a pant, an adult incontinence product, a sanitary napkin or any other article that may benefit from having at least a portion thereon that is elastically stretchable. In one embodiment, ears or side panels may be cut from such a stretchable laminate and one side edge of the ear or side panel may be joined to the chassis of a disposable absorbent article. A disposable absorbent article 70 that include a back waist region 170, a crotch region 270, and a front waist region 370 is schematically represented in FIG. 13. A pair of ears 75 or side panels formed at least partially of the stretchable laminate are attached along their respective proximal edge to the left and right sides of the disposable absorbent article 70 respectively. A fastener 74 such as a mechanical fastener comprising a plurality of extending hooks or an adhesive may be connected to a portion of the ear or side panel about the distal edge of the ear or side panel. Such a fastener 74 may in combination with the stretchable laminate provide for proper placement and attachment of the absorbent article about the lower torso of a wearer. The fasteners 74 may be engaged with a landing zone 76 in the front waist region. In another embodiment, any such stretchable laminate may be used as an integral outer cover or backsheet for an absorbent article.

If the stretchable laminates of the present disclosure are used as ears 75 or side panels of an absorbent article, they may be joined to the absorbent article 70 such that the machine direction of the stretchable laminates or nonwoven substrates thereof is generally parallel to a longitudinal axis “Long A” of the absorbent article 70. Nonwoven layers or nonwoven substrates in the stretchable laminates are more resistant to tensile forces in the machine direction when compared to tensile forces applied about their cross-direction. As a result, it is desired to position the stretchable laminates on the absorbent article such that the user or caregiver expands the ear 75 or side panel in a cross-direction when donning the absorbent article to allow for easier expansion of the stretchable laminates.

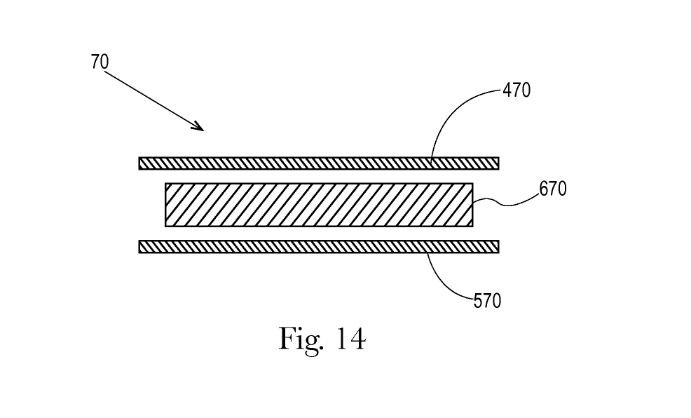

Referring to FIG. 14, a typical chassis of a disposable absorbent article 70 may include a liquid pervious layer or top sheet 470, a liquid impervious layer or backsheet 570, and an absorbent core 670 disposed between the liquid pervious layer 470 and the liquid impervious layer 570. The ears 75 or side panels formed at least partially of the stretchable laminates of the present disclosure may be joined to any of the liquid pervious layer 470, the liquid impervious layer 570 and/or the absorbent core 670. The ears 75 or side panels may also be joined to other portions of the absorbent article 70. The absorbent article may also include any features that may be suitable for such an absorbent article and are generally known in the art, such as leg cuffs, for example.

In an embodiment, an absorbent article may comprise a liquid pervious layer, a liquid impervious layer, an absorbent core disposed between the liquid pervious layer and the liquid impervious layer. The absorbent article may also comprise a stretchable laminate joined to any of the liquid pervious layer, the liquid impervious layer, and the absorbent core. The stretchable laminate may be any stretchable laminate of the present disclosure. In some embodiments, the stretchable laminate may comprise a nonwoven substrate comprising a layer of spunbond fibers, wherein a plurality of, or all of, the spunbond fibers are formed from a composition comprising a polyolefin and an inorganic filler. The inorganic filler may be present in the composition at a level of between about 5% and about 20% by weight of the composition or at any other levels disclosed herein. The stretchable laminate may also comprise an elastomeric material joined to a side or first side of the nonwoven substrate. The stretchable laminate may comprise a second nonwoven substrate joined to the elastomeric material on an opposite side as the nonwoven substrate. The second nonwoven substrate may comprise a layer of carded fibers, the inorganic filler may comprise an alkaline carbonate, and the polyolefin may comprise polypropylene. In some embodiments, the second nonwoven substrate may comprise a layer of spunbond fibers, wherein a plurality of the spunbond fibers are formed from a composition comprising a thermoplastic polymer and an inorganic filler, and wherein the inorganic filler may be present in the composition at a level of between about 5% and about 15% or between about 3% and about 20% by weight of the composition, for example. The stretchable laminate may form at least a portion of, or all of, a side panel, outer cover, or ear of the absorbent article. The nonwoven substrate and the second nonwoven substrate may comprise meltblown layers. The meltblown layers may not comprise any inorganic fillers and may comprise less than 10%, less than 5%, or less than 1% inorganic fillers, by weight.

In an embodiment, an absorbent article of the present disclosure may comprise a liquid pervious layer, a liquid impervious layer, an absorbent core disposed between the liquid pervious layer and the liquid impervious layer, and two side panels or ears joined to any of the liquid pervious layer, the liquid impervious layer, and the absorbent core. One or both of the side panels or ears may comprise a first nonwoven substrate, a second nonwoven substrate, and an elastomeric material disposed intermediate the first nonwoven substrate and the second nonwoven substrate. One or both of the side panels or ears may comprise a fastener element 74 (see FIG. 13), such as a portion of a hook and loop fastener. The fastener element may be engagable with a landing zone 76 (see FIG. 13) when the absorbent article is donned on a wearer. The first nonwoven substrate may comprise a layer of spunbond fibers. A plurality of the spunbond fibers may be formed from a composition comprising a polyolefin and an inorganic filler. The inorganic filler may be present in the composition at a level of between about 5% and about 20%, between about 9% and about 13%, or about 11.5% by weight of the composition. The first nonwoven substrate may further comprise a layer of meltblown fibers that are free of, or substantially free of, the inorganic filler. The second nonwoven substrate may comprise a layer of spunbond fibers. A plurality of the spunbond fibers may be formed from a composition comprising a polyolefin and an inorganic filler. The inorganic filler may be present in the composition at a level of between about 3% and about 25%, between about 9% and 13%, or about 11.5% by weight of the composition. The second nonwoven substrate may further comprise a layer of meltblown fibers. The meltblown fibers may be free of, or substantially free of, the inorganic filler.

In an embodiment, an absorbent article of the present disclosure may comprise a liquid pervious layer, a liquid impervious layer, an absorbent core disposed between the liquid pervious layer and the liquid impervious layer, and two side panels or ears joined to any of the liquid pervious layer, the liquid impervious layer, and the absorbent core. One or both of the side panels or ears may comprise a first nonwoven substrate, a second nonwoven substrate, and an elastomeric material disposed intermediate the first nonwoven substrate and the second nonwoven substrate. One or both of the side panels or ears may comprise a fastening element 74 (see FIG. 13), such as a portion of a hook and loop fastener. The fastening element may be engagable with a landing zone 76 (see FIG. 13) when the absorbent article is donned on a wearer. The first nonwoven substrate may comprise a layer of spunbond fibers. A plurality of the spunbond fibers may be formed from a composition comprising a polyolefin and an inorganic filler. The inorganic filler may be present in the composition at a level of between about 5% and about 20%, between about 9% and about 13%, or about 11.5% by weight of the composition. The first nonwoven substrate may further comprise a layer of meltblown fibers that are free of, or substantially free of, the inorganic filler. The second nonwoven substrate may comprise a layer of carded fibers.

In an embodiment, a method of making a stretchable laminate for an absorbent article is provided by the present disclosure. The method may comprise providing a nonwoven substrate comprising a layer of spunbond fibers. A plurality of, or all of, the spunbond fibers may be formed from a composition comprising a polyolefin and an inorganic filler. The inorganic filler may be present in the composition at a level of between 5% and 15% or between 3% and 25% by weight of the composition. The method may further comprise providing an elastomeric material, and joining the nonwoven substrate to a side or first side of the elastomeric material. The method may further comprise providing a second nonwoven substrate, and joining the second nonwoven substrate to a second side of the elastomeric material. In an embodiment, the second nonwoven substrate may comprise a layer of carded fibers. In some embodiments, the second nonwoven substrate may comprise a layer of spunbond fibers. A plurality of, or all of, the spunbond fibers are formed from a composition comprising a thermoplastic polymer and an inorganic filler. The inorganic filler may be present in the composition at a level of between 5% and 15% or between 3% to 25% by weight of the composition.

In an embodiment, a 17 gsm basis weight SSMMS nonwoven substrate used in the stretchable laminates of the present disclosure may comprise about 3.0 gsm of meltblown layers without any inorganic fillers in the meltblown fibers, and about 14 gsm of spunbond layers formed from a composition comprising about 11.5% calcium carbonate or other inorganic filler by weight of the composition. The overall percentage of calcium carbonate or other inorganic filler in the SSMMS is about 9.5%. Both the meltblown fibers and the spunbond fibers may comprise a thermoplastic polymer, such as a polyolefin.

In an embodiment, a 14 gsm basis weight SSMMS nonwoven substrate used in the stretchable laminate of the present disclosure may comprise about 2.4 gsm of meltblown layers without any inorganic fillers in the meltblown fibers, and about 11.6 gsm of spunbond layers formed from a composition comprising about 11.5% calcium carbonate or other inorganic filler by weight of the composition. The overall percentage of calcium carbonate or other inorganic filler in the SSMMS is about 9.47%. Both the meltblown fibers and the spunbond fibers may comprise a thermoplastic polymer, such as a polyolefin.

In an embodiment, a 20 gsm basis weight SSMMS nonwoven substrate used in the stretchable laminates of the present disclosure may comprise about 3.0 gsm of meltblown layers without any inorganic fillers in the meltblown fibers, and about 17 gsm of spunbond layers formed from a composition comprising about 11.5% calcium carbonate or other inorganic filler by weight of the composition. The overall percentage of calcium carbonate or other inorganic filler in the SSMMS is about 9.78%. Both the meltblown fibers and the spunbond fibers may comprise a thermoplastic polymer, such as a polyolefin.

The nonwoven substrates of the present disclosure may have inorganic material present therein, by weight of the nonwoven substrate, in the ranges of between 1% and 50%, between 2% and 40%, between 3% and 30%, between 4% and 25%, between 5% and 20%, between 3% and 20%, between 5% and 15%, between 3% and 15%, between 5% and 12%, between 6% and 12%, between 8% and 12%, between 8% and 11%, between 8% and 10%, specifically reciting all 0.5% increments within the above-specified ranges. The nonwoven substrates of the present disclosure may also have inorganic material present therein, by weight, of about 7%, about 8%, about 9%, about 10%, about 11%, or about 12%. The inorganic material may be present in the meltblown fibers, the spunbond fibers, or both the meltblown and spunbond fibers. Not all meltblown or spunbond layers may have inorganic material within fibers thereof. The weight percentages of inorganic material in a nonwoven substrate can be measured using the Ash Test herein below.

In an embodiment, the laminate may not be stretchable and may not comprise an elastomeric film. The laminate may comprise one or more spunbond layers comprising the inorganic fillers described herein and one or more spunbond layers optionally comprising the inorganic fillers described herein. The laminate may also include other materials or layers.

In an embodiment, a stretchable laminate for an absorbent article may comprise a first nonwoven substrate comprising two layers of spunbond fibers and a layer of meltblown fibers disposed between the two layers of spunbond fibers. A plurality of the spunbond fibers in each of the spunbond layers may be formed from a composition comprising a thermoplastic polymer and an inorganic filler. The inorganic filler may be present in the composition at a level of between 3% and 20% by weight of the composition or between 5% and 15% by weight of the first nonwoven substrate. The stretchable laminate may comprise a second nonwoven substrate comprising two layers of spunbond fibers and a layer of meltblown fibers disposed between the two layers of spunbond fibers. A plurality of the spunbond fibers in each of the spunbond layers may be formed from a composition comprising a thermoplastic polymer and an inorganic filler. The inorganic filler may be present in the composition at a level of between 3% and 20% by weight of the composition or between 3% and 15% by weight of the second nonwoven substrate. The stretchable laminate may comprise one or more elastomeric materials that may be disposed between the first nonwoven substrate and the second nonwoven substrate.

The meltblown fibers in the first and second nonwoven substrate may or may not comprise one or more inorganic fillers. In other embodiments, only some of the meltblown fibers may comprise one or more inorganic fillers. The one or more inorganic fillers in the meltblown fibers may be the same as or different than (e.g., different average or maximum particle size, different inorganic filler) the inorganic filler in the spunbond fibers. The average particle size of the inorganic filler in the meltblown fibers may be smaller than the average particle size of the inorganic filler in the spunbond fibers of the first or second nonwoven substrates. The layer of meltblown fibers in the first or second nonwoven substrates may comprise less inorganic filler, by weight, than each of the layers of the spunbond fibers in the first or second nonwoven substrates. The fibers in the first spunbond layer of the first or second nonwoven substrates may have a greater percentage, by weight, of the inorganic filler as the fibers in the second spunbond layer of the first or nonwoven substrates. The spunbond fibers in the first or second nonwoven substrates may have a density greater than, less than, or about equal to the density of the meltblown fibers in the first or second nonwoven substrates. The meltblown fibers in the second nonwoven substrate may comprise less or more inorganic filler by weight than the spunbond fibers in the first or second nonwoven substrates.

In an embodiment, a stretchable laminate for an absorbent article may comprise a first nonwoven substrate comprising two layers of spunbond fibers and a layer of meltblown fibers disposed between the two layers of spunbond fibers. A plurality of the spunbond fibers may be formed from a composition comprising a thermoplastic polymer and an inorganic filler. The inorganic filler may be present in the composition at a level of between 3% and 20% by weight of the composition or between 5% and 15% by weight of the first nonwoven substrate. The stretchable laminate may comprise a second nonwoven substrate and elastomeric material disposed between the first nonwoven substrate and the second nonwoven substrate. The second nonwoven substrate may comprise a layer of carded fibers. The carded fibers may comprise one or more inorganic fillers in any suitable weight percentage. The meltblown fibers in the first or second nonwoven substrates may comprise a second inorganic filler that is different than the inorganic filler in the spunbond fibers. The second inorganic filler may have a smaller average particle size than the average particle size of the inorganic filler. The density of the fibers of the first spunbond layer may be about the same as, less than, or greater than, the density of the fibers in the second spunbond layer.

In an embodiment, referring to FIG. 1, the spunbond layer 120 may comprise fibers formed from a composition comprising one or more inorganic fillers and one or more thermoplastic polymers. The inorganic fillers may be present in the composition in the range of 5% to 20% by weight of the composition. Fibers in the spunbond layer 320 (the outer layer) may be free of inorganic fillers. In another embodiment, fibers in the spunbond layer 320 may be formed from a composition comprising less than 5%, less than 3%, or less than 1% of inorganic fillers by weight of the composition. In such embodiments, the spunbond layers with the most or all of the inorganic fillers are positioned internal to the stretchable laminate 10 to thereby reduce or prevent absorbent article manufacturing equipment contamination and degradation that may be caused by the inorganic fillers in the outer spunbond layers. Stated another way, an outer spunbond layer or other layer may be free of or substantially free of inorganic fillers to reduce absorbent article manufacturing equipment contamination and degradation that may be caused by the inorganic fillers.

In an embodiment, referring to FIG. 2, the spunbond layer 320 may be free of or substantially free (e.g., less than 3% or less than 1%) of one or more inorganic fillers, while the spunbond layers 1220 and 2120 may comprise one or more inorganic fillers (in the same weight percents or different weight percents as described above with respect to the spunbond layer 120). In an embodiment, referring to FIG. 3, the spunbond layers 320 and 340 may be free of or substantially free of one or more inorganic fillers, while the spunbond layers 2120, 1120, 1140, and 2140 may comprise one or more inorganic fillers (in the same weight percents or different weight percents as described above with respect to the spunbond layer 120). Again, these embodiments may be utilized to reduce absorbent article manufacturing equipment degradation or contamination that may be caused by inorganic fillers within outer spunbond layers.

More generally, to reduce or prevent absorbent article manufacturing equipment contamination or degradation that may be caused by the inorganic fillers disclosed herein, the outer surfaces or layers of the nonwoven substrates and/or stretchable laminates of the present disclosure may comprise little (e.g., less than 3% or less than 1%) or no inorganic fillers, while internal layers (including meltblown layers) of the nonwoven substrates and/or stretchable laminates may comprise one or more inorganic fillers.

In some embodiments, the present disclosure provides activated or mechanically activated nonwoven substrates for use in absorbent articles, such as sanitary napkins, taped diapers, or pant diapers, for example. The nonwoven substrates, in some embodiments, may function as topsheets, backsheets, apertured topsheets, acquisition layers, portions of wings of sanitary napkins, and portions of side panels, ears, and/or fasteners of diapers, for example. Other uses on other nonwoven substrate containing products are also envisioned and are within the scope of the present disclosure.

In an embodiment, an absorbent article, such as a taped diaper, pant, or sanitary napkin, for example, may comprise an activated nonwoven substrate that may comprise one or more spunbond layers, such as two or three spunbond layers, for example. Each of the spunbond layers may comprise a plurality of fibers formed from a composition comprising an inorganic filler and a thermoplastic polymer. The inorganic filler may be present in the composition in the range of about 5% to about 20% or about 5% to about 15% by weight of the composition or may be present in the nonwoven substrate in the range of about 3% to about 20% or about 3% to about 15% by weight of the nonwoven substrate. Each 0.5% increment within the above-specified ranges is specifically disclosed herein. The nonwoven substrates may also optionally comprise one or more meltblown layers. In some embodiment, the activated nonwoven substrate may form a portion of or all of a topsheet of the absorbent article. The topsheet may be apertured. The activated nonwoven substrate may form a portion of or all of a wing of a sanitary tissue product. The absorbent article may also comprise an absorbent core, a backsheet, and acquisition layer, ears, and/or any other components typically provided on absorbent articles.

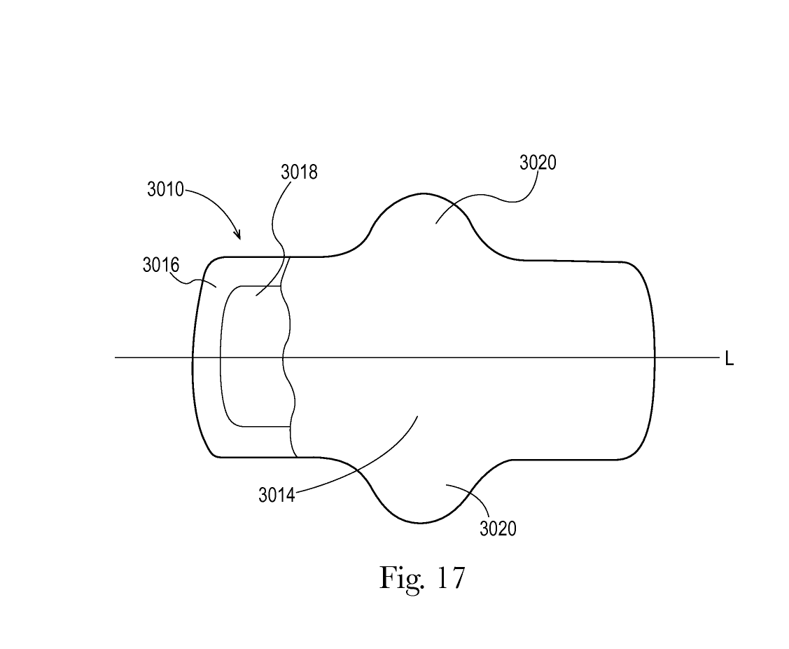

In an embodiment, referring to FIG. 17, the absorbent article may be a sanitary napkin 3010. The sanitary napkin 3010 may comprise a topsheet 3014, a backsheet 3016, and an absorbent core 3018. The sanitary napkin 3010 may also comprise wings 3020 extending outwardly with respect to a longitudinal axis, L, of the sanitary napkin 3010. The wings 3020 may be joined to the topsheet 3014, the backsheet 3016, and/or the absorbent core 3018. The stretchable laminates and/or nonwoven substrates of the present disclosure may be used in the topsheet 3014, the backsheet 3016, components of the absorbent core 3018, the wings 3020, and/or in other components of the sanitary napkin 3010. The sanitary napkin 3010 may also be provided with additional features commonly found in sanitary napkins as is known in the art.

In an embodiment, the elastomeric materials or films may also comprise one or more inorganic fillers, as the inorganic fillers are described herein. The elastomeric materials may comprise the inorganic fillers at a weight % of 1% to 99%, 1% to 50%, 1% to 25%, 1% to 10%, specifically including all 0.5 percent increments within the above-recited ranges. In some embodiments, a stretchable laminate may comprise at least one spunbond layer comprising one or more inorganic fillers and at least one elastomeric material comprising one or more inorganic fillers. The stretchable laminate may also comprise at least one meltblown layer either comprising inorganic fillers or being free of any inorganic fillers. The inorganic fillers may be the same or different in each material and may be present at the same weight percent or at different weight percent in each material. In an embodiment, a stretchable laminate may comprise one or more inorganic fillers only in its elastomeric material and not in its spunbond or meltblown layers.

### EXAMPLES

### Comparative Example 1

An embodiment of the stretchable laminate of the present disclosure was tested and various properties were compared to a conventional stretchable laminate not having any CaCO₃ within fibers of spunbond layers of the nonwoven substrate.

The stretchable laminate of the present disclosure that was tested had the following layers bonded together to form the laminate:

A 24 gsm carded fiber layer

A 50 gsm elastomeric film

A 14 gsm SSMMS nonwoven (S=spunbond and M=Meltblown)
Each of the S layers were formed from a composition comprising polypropylene and about 11.5% CaCO₃, by weight, of the composition
Each of the M layers were formed from polypropylene and did not have any CaCO₃ additive

The conventional stretchable laminate that was tested had the following layers bonded together to form the laminate:

A 24 gsm carded fiber layer

A 50 gsm elastomeric film

A 14 gsm SSMMS nonwoven
none of the S or M layers contained any CaCO₃ additive
both the S and M layers were formed from fibers comprising polypropylene

In essence, a 14 gsm SSMMS nonwoven substrate with additive vs. a 14 gsm SSMMS nonwoven substrate without additive was tested to determine how the additive would affect the properties of the stretchable laminates.

After formation of both of the stretchable laminates, various properties of both laminates were tested after mechanical activation to certain DOEs. The DOEs are D1, D2, D3, and D4 all of which are different. A vision system from Optical Control Systems, GmbH (“OCS”) was used to determine the number of holes extending through the stretchable laminate, specifically holes through all layers and elastomeric materials of the laminates. Physical testing (i.e., human examination of the stretchable laminates) was also used to determine the number of holes. The results of the hole count testing appears in the chart below. As is seen in the chart, the number of holes in the stretchable laminates of the present disclosure is significantly less than the number of holes in the conventional stretchable laminates owing to the CaCO₃ additive within the fibers of the spunbond layers of the stretchable laminates of the present disclosure. Furthermore, fibers with the inorganic fillers are denser than fibers without the inorganic fillers. As such, a nonwoven substrate comprising a spunbond layer having fibers formed from a composition comprising the inorganic fillers will have less fibers than a nonwoven substrate without a spunbond layer having fibers formed from a composition comprising the inorganic fillers, when each of the nonwoven substrates have the same basis weight. This can provide cost savings.

A Max Peak Force Test and a 1000 gram Extension Test were also run on the stretchable laminates. Those tests are described in further detail below. The results of the Max Peak Force Test and the 1000 gram Extension Test appear in the chart below. As can be seen, even though the stretchable laminate of the present disclosure had 11.5% CaCO₃ additive, by weight, in the fibers in its spunbond layers, the Maximum Peak Force and the 1000 gram Extension data remained about the same. This indicates that even with the CaCO₃ additive, the stretchable laminates of the present disclosure are able to achieve strengths almost identical to conventional stretchable laminates.

It is believed that the CaCO₃ in the spunbond fibers allows the fibers to become more stretchable without actually breaking during mechanical activation. A higher Max Peak Force can be translated into a higher risk of creating holes in the stretchable laminate during mechanical activation. Once the nonwoven substrate starts to break apart during mechanical activation, the force is transferred onto the elastomeric film. Thus, the higher the force or energy transferred to the elastomeric film, the higher the risk of creating holes in the elastomeric film. This leads to the conclusion that by providing CaCO₃ in spunbond fibers, the nonwoven substrates are more mechanical activation friendly compared to nonwoven substrates without the CaCO₃ in spunbond fibers at the same glue add on rate and DOE.

| Hole Count | | | | | | | | |
|---|---|---|---|---|---|---|---|---|
| | | | Present | | | | | |
| | Conventional | | Disclosure | | Technical Data | | | |
| | OCS | | OCS | | Conventional | | Present Disclosure | |
| | Vision | Physical | Vision | Physical | | 1000 g | | 1000 g |
| DOE | System | Testing | System | Testing | MPF | Extension | MPF | Extension |
| D1/D2 | 38 | 1 | 14 | 0 | 54.9 | 42.9 | 51.2 | 42.3 |
| D1/D3 | 3 | 5 | 0 | 0 | 54.0 | 42.5 | 52.6 | 41.6 |
| D4/D1 | 5 | 2 | 2 | 1 | 50.9 | 39.1 | 50.7 | 39.9 |
| D4/D4 | 14 | 1 | 9 | 0 | 49.4 | 38.7 | 54.8 | 38.0 |

The stretchable laminates of the present disclosure may also be used to achieve higher DOEs to provide extension properties better than conventional stretchable laminates while still achieving about the same number of holes that appear in conventional stretchable laminates. Stated another way, instead of reducing the number of holes, the stretchable laminates may be used instead to allow for greater extension while incurring about the same number of holes as the conventional stretchable laminates.

### Comparative Example 2

An embodiment of the stretchable laminate of the present disclosure was tested and various properties were compared to a conventional stretchable laminate not having any CaCO₃ additive within fibers of spunbond layers of the nonwoven substrate.

The stretchable laminate of the present disclosure that was tested had the following layers bonded together to form the laminate:

A 20 gsm SSMMS nonwoven
Each of the S layers were formed from a composition comprising polypropylene and about 11.5% CaCO₃, by weight, of the composition
Each of the M layers were formed from polypropylene without any CaCO₃ additive

A 50 gsm elastomeric film

A 14 gsm SSMMS nonwoven
Each of the S layers were formed from a composition comprising polypropylene and about 11.5% CaCO₃, by weight, of the composition
Each of the M layers were formed from polypropylene without any CaCO₃ additive

The conventional stretchable laminate that was tested had the following layers bonded together to form the laminate:

A 20 gsm SSMMS nonwoven
none of the S or M layers contained any CaCO₃ additive
both the S and M layers were formed from fibers comprising polypropylene

A 50 gsm elastomeric film

A 14 gsm SSMMS nonwoven
none of the S or M layers contained any CaCO₃ additive
both the S and M layers were formed from fibers comprising polypropylene

After formation of both of the stretchable laminates, various properties of both laminates were tested after mechanical activation to certain DOEs. The DOEs are D1, D2, D3, D4, and D5, all of which are different. D1 through D4 are the same as Example 1 above. A vision system from Optical Control Systems, GmbH (“OCS”) was used to determine the number of holes extending through the stretchable laminate. Physical testing (i.e., human examination of the stretchable laminates) was also used to determine the number of holes. The results of the hole count testing appears below. As is seen in the chart, the number of holes in the stretchable laminates of the present disclosure is significantly less than the number of holes in the conventional stretchable laminates owing to the CaCO₃ filler in the fibers of the spunbond layers. Furthermore, fibers with the inorganic fillers are denser than fibers without the inorganic fillers. As such, a nonwoven substrate comprising a spunbond layer having fibers formed from a composition comprising the inorganic fillers will have less fibers than a nonwoven substrate without a spunbond layer having fibers formed from a composition comprising the inorganic fillers, when each of the nonwoven substrates have the same basis weight. This can provide cost savings.

A Max Peak Force Test and a 1000 gram Extension Test were also run on the stretchable laminates. Those tests are described in further detail below. The result of the Max Peak Force Test and the 1000 gram Extension Test appear in the chart below. As can be seen, even though the stretchable laminates of the present disclosure had 11.5% CaCO₃ additive, by weight, in the fibers in its spunbond layers, the Maximum Peak Force and the 1000 gram Extension data remained about the same. This indicates that even with the CaCO₃ additive, the stretchable laminates of the present disclosure are able to achieve strengths almost identical to conventional stretchable laminates.

It is believed that the CaCO₃ in the spunbond fibers allows the fibers to become more stretchable without actually breaking during mechanical activation. A higher Max Peak Force can be translated into a higher risk of creating holes in the stretchable laminate during mechanical activation. Once the nonwoven substrate starts to break apart during mechanical activation, the force is transferred to the elastomeric film. Thus, the higher the force or energy transferred to the elastomeric film, the higher the risk of creating holes in the elastomeric film. This leads to the conclusion that by providing CaCO₃ in spunbond fibers, the nonwoven substrates are more mechanical activation friendly compared to nonwoven substrates without the CaCO₃ in spunbond fibers at the same glue add on rate and DOE.

| Hole Count | | | | | | | | |
|---|---|---|---|---|---|---|---|---|
| | | | Present | | | | | |
| | Conventional | | Disclosure | | Technical Data | | | |
| | OCS | | OCS | | Conventional | | Present Disclosure | |
| | Vision | Physical | Vision | Physical | | 1000 g | | 1000 g |
| DOE | System | Testing | System | Testing | MPF | Extension | MPF | Extension |
| D1/D5 | 157 | 24 | 27 | 6 | 47.8 | 41.2 | 47.0 | 41.8 |
| D1/D2 | 103 | 28 | 5 | 5 | 53.6 | 39.3 | 52.0 | 39.3 |
| D4/D3 | 14 | 13 | 8 | 4 | 47.9 | 37.7 | 48.3 | 37.5 |

The stretchable laminates of the present disclosure may also be used to achieve higher DOEs to provide extension properties better than conventional stretchable laminates while still achieving about the same number of holes that appear in conventional stretchable laminates. Stated another way, instead of reducing the number of holes, the stretchable laminates may be used instead to allow for greater extension while incurring about the same number of holes as the conventional stretchable laminates.

### Comparative Example 3

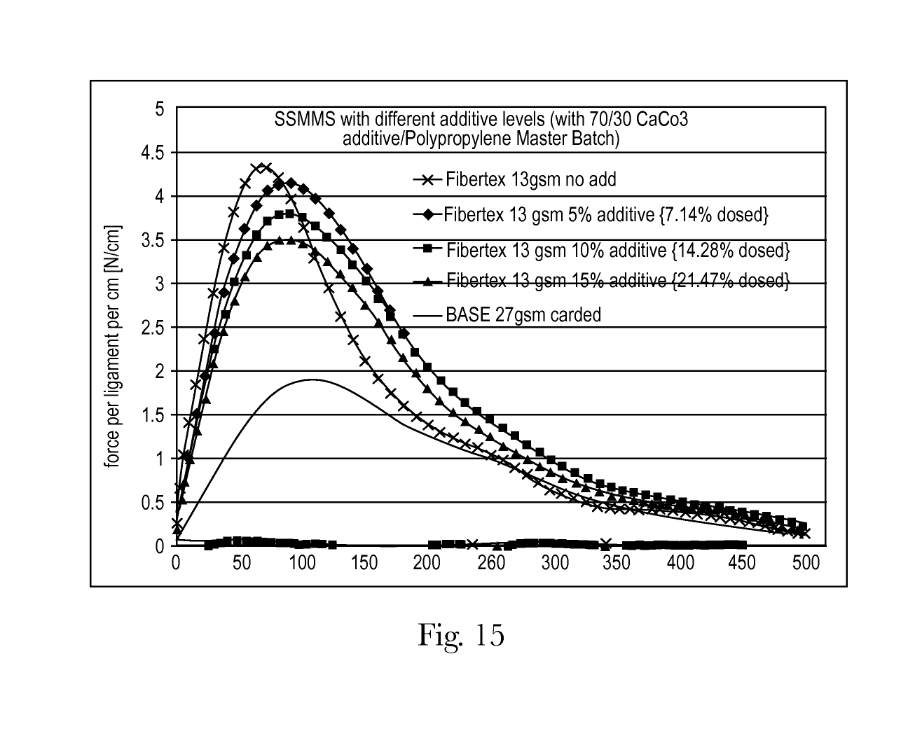

Referring to FIG. 15, a ring rolling simulation apparatus and method was used to determine the Max Force per ligament per N/cm and the Strain at Max Force % for each of the SSMMS nonwoven substrates (no elastomeric material or film) and the base carded fiber material tested. The ring rolling simulation apparatus and method simulates mechanical activation of a material in the cross direction and is disclosed in detail in U.S. Pat. Nos. 6,843,134, 7,024,939, and 7,062,983. The data of FIG. 15 is taken in the cross direction. The SSMMS nonwoven substrates tested were a conventional Fibertex 13 gsm SSMMS nonwoven substrate with no CaCO₃ additive in the spunbond layers, a Fibertex 13 gsm SSMMS nonwoven substrate with 5% CaCO₃ additive in the spunbond layers, a Fibertex 13 gsm SSMMS nonwoven substrate with 10% CaCO₃ additive in the spunbond layers, a Fibertex 13 gsm SSMMS nonwoven substrate with 15% CaCO₃ additive in the spunbond layers, and a conventional 27 gsm carded fiber material. The meltblown fibers did not have any CaCO₃ additive. The results appear in the chart below.

| Laminate | | |
|---|---|---|
| Composition | Max Force N/CM | Strain at Max Force % |
| 13 gsm No | 4.33 | 71.31 |
| CaCO₃ additive | | |
| 13 gsm 5% | 4.14 | 90.29 |
| CaCO₃ additive | | |
| 13 gsm 10% | 3.79 | 90.30 |
| CaCO₃ additive | | |
| 13 gsm 15% | 3.49 | 90.31 |
| CACO₃ additive | | |

As can be deduced from the data, the Max Force is lower and is achieved at a higher Strain % when more CaCO₃ additive is within the fibers of the spunbond layers of the SSMMS nonwovens when compared to a conventional 13 gsm SSMMS nonwoven without any CaCO₃ additive in the spunbond layers. A higher Max Force can be translated into a higher risk of creating holes during mechanical activation in that once the nonwoven substrate starts to break, the force is transferred to the elastomeric material or film. Additionally of note is the gradual slope of the graphs of the SSMMS nonwoven substrates of the present disclosure after the Max Force is achieved. It is believed that this gradual slope indicates slower transfer of energy (due to fiber breakage during mechanical activation) to the elastomeric material or film of a stretchable laminate, thereby creating less holes in the elastomeric material or film during mechanic activation.

### Comparative Example 4

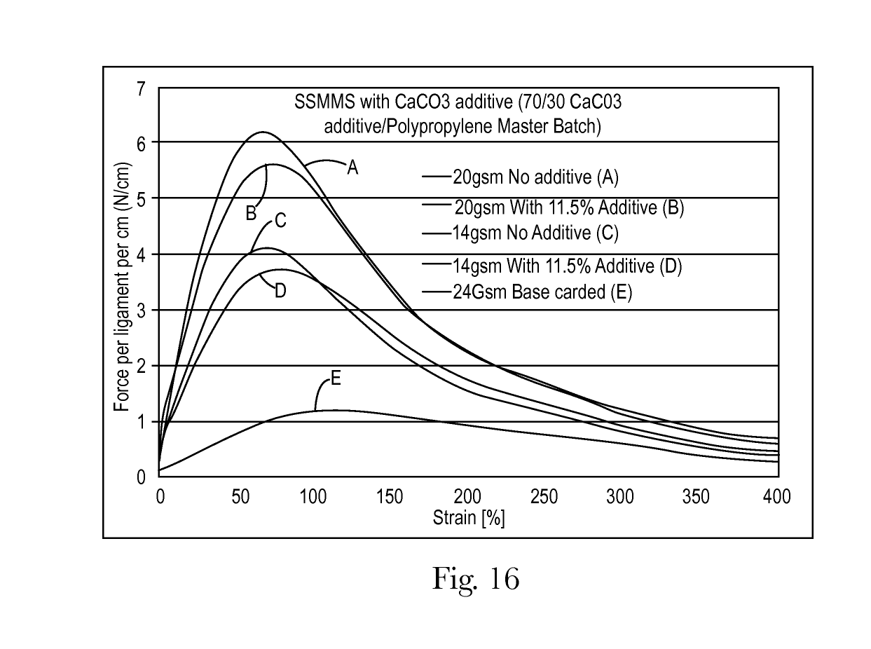

Referring to FIG. 16, a ring rolling simulation apparatus and method was used to determine the Max Force per ligament per N/cm and the Strain at Max Force % for each of the SSMMS nonwoven substrates (no elastomeric material or film) and the base carded fiber tested. The ring rolling simulation apparatus and method simulates mechanical activation of a material in the cross direction and is disclosed in detail in U.S. Pat. Nos. 6,843,134, 7,024,939, and 7,062,983. The data of FIG. 16 is taken in the cross direction. The SSMMS nonwoven substrates tested were a conventional; Fibertex 20 gsm SSMMS nonwoven substrate with no CaCO₃ additive (labeled A in the FIG. 16) in the spunbond layers, a Fibertex 20 gsm SSMMS nonwoven substrate with 11.5% CaCO₃ additive in the spunbond layers (labeled B in the FIG. 16), a conventional Fibertex 14 gsm SSMMS nonwoven substrate with no CaCO₃ additive in the spunbond layers (labeled C in the FIG. 16), a Fibertex 14 gsm SSMMS nonwoven substrate with 11.5% CaCO₃ additive in the spunbond layers (labeled D in the FIG. 16), and a conventional 27 gsm carded fiber material (labeled E in the FIG. 16).

| | Max Force N/CM | Strain at max Force % |
|---|---|---|
| 20 gsm No additive | 6.17 | 62.56 |
| 20 gsm 11.5% | 5.64 | 71.78 |
| CaCO₃ | | |
| 14 gsm No additive | 4.10 | 71.8 |
| 14 gsm 11.5% | 3.74 | 81.21 |
| CaCO₃ | | |

As can be deduced from the data, the Max Force is lower and is achieved at a higher Strain % when more CaCO₃ additive is within the fibers of the spunbond layers of the SSMMS nonwoven substrates when compared to a conventional 13 gsm SSMMS nonwoven substrate without any CaCO₃ additive. A higher Max Force can be translated into a higher risk of creating holes during mechanical activation in that once the nonwoven substrate starts to break, the force is transferred to the elastomeric material or film. Additionally of note is the gradual slope of the graphs of the SSMMS nonwoven substrates of the present disclosure after the Max Force is achieved. It is believed that this gradual slope indicates slower transfer of energy (due to fiber breaking during mechanical activation) to the elastomeric material or film of a stretchable laminate, thereby creating less holes in the elastomeric material or film during mechanic activation.

### Methods

All samples are preconditioned at about 23° C.±2° C. and about 50%±2% relative humidity for 2 hours prior to testing.

### Maximum Peak Force/1000 Gram Extension

Maximum Peak Force and 1000 Gram Extension at 9.81 N Force are measured on a constant rate of extension tensile tester with a computer interface (a suitable instrument is the MTS Insight using Testworks 4.0 Software, as available from MTS Systems Corp., Eden Prairie, Minn.) using a load cell for which the forces measured are within 10% to 90% of the limit of the load cell. The movable (upper) pneumatic jaw is fitted with a set of a flat rubber faced grip with matching contact line grip which is 25.4 mm wide. The stationary (bottom) pneumatic jaw is fitted with a pair of 25.4 mm rubber faced grips wider than the width of the test specimen. Air pressure supplied to the jaws should be sufficient to prevent sample slippage. All testing is performed in a conditioned room maintained at about 23° C.±2° C. and about 50° C.±2° C. relative humidity.

Remove a stretchable laminate from a chassis of an absorbent article. Using a digital micrometer (traceable to NIST or other standards organization), measure the lateral width of the stretchable laminate from the laminate/chassis bond to the proximal edge of the attachment tab, and record to the nearest 1.0 mm. This distance, L1, should encompass the stretchable region of the stretchable laminate.

Set the gage length to L1. Zero the crosshead and the load cell. Insert the tab end or fastener end of the specimen into the upper grips and align the proximal edge of the specimen's tab with the horizontal center of the grip. With the specimen aligned vertically within the upper and lower jaws, close the upper grips. Insert the chassis end of the specimen into the lower grips and close the lower grips. The specimen should be under enough tension to eliminate any slack, but less than 0.05 N of force on the load cell.

Program the tensile tester to perform an extension test, collecting force and extension data at an acquisition rate of 50 Hz as the crosshead raises at a rate of 508 mm/min until a force of 10 N is reached, after which the crosshead is returned to its original position. Start the tensile tester and data collection. Program the software to calculate the Maximum Peak Force and the 1000 Gram Extension at 9.81 N force from the constructed force (N) verses extension (mm) curve.

Report the Maximum Peak Force to the nearest 0.01 N and the 1000 Gram Extension at 9.8 N Force to the nearest 0.01 mm, and record the results. Repeat the test and record the results for 10 replicate samples. Calculate and report the average Maximum Peak Force to the nearest 0.01 N and the average 1000 Gram Extension at 9.8 N Force to the nearest 0.01 mm.

### Determining %, by Weight, of Inorganic Material in Nonwoven Substrates

By measuring the weight of the ash when a nonwoven substrate is burned, it is possible to determine the amount of inorganic material in the nonwoven substrate. The organic material will be burned away leaving only the inorganic material behind.

Samples of the nonwoven substrate can be obtained individually or may be separated from an absorbent article.

The ASTM method designated as D5630-06, Procedure A, is used to determine the Ash % and, thereby, the % inorganic material, by weight, in a nonwoven substrate sample.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any embodiment disclosed or claimed herein or that it alone, or in any combination with any other reference or references, teaches, suggests or discloses any such embodiment. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications may be made without departing from the spirit and scope of the present disclosure. It is therefore intended to cover in the appended claims all such changes and modifications that are within the scope of this disclosure.

What is claimed is:

1. A stretchable laminate comprising:
a nonwoven substrate comprising a layer of spunbond fibers, wherein a plurality of the spunbond fibers are formed from a composition comprising a thermoplastic polymer and an inorganic filler, and wherein the inorganic filler is present in the nonwoven substrate at a level of between 1% and 20% by weight of the nonwoven substrate; and
an elastomeric material, wherein the nonwoven substrate is joined to a side of the elastomeric material.

2. The stretchable laminate of claim 1, wherein the thermoplastic polymer comprises a polyolefin.

3. The stretchable laminate of claim 2, wherein the polyolefin comprises polypropylene.

4. The stretchable laminate of claim 1, comprising a second nonwoven substrate joined to a second side of the elastomeric material such that the elastomeric material is disposed at least partially between the nonwoven substrate and the second nonwoven substrate.

5. The stretchable laminate of claim 4, wherein the second nonwoven substrate comprises a layer of carded fibers.

6. The stretchable laminate of claim 4, wherein the second nonwoven substrate comprises a layer of spunbond fibers.

7. The stretchable laminate of claim 6, wherein a plurality of the spunbond fibers of the second nonwoven substrate are formed from the composition comprising the thermoplastic polymer and the inorganic filler, and wherein the inorganic filler is present in the second nonwoven substrate at a level of between 3% and 15% by weight of the nonwoven substrate.

8. The stretchable laminate of claim 1, wherein the elastomeric material comprises an elastic film.

9. The stretchable laminate of claim 1, wherein the nonwoven substrate comprises:
a layer of meltblown fibers free of any inorganic fillers; and
a second layer of spunbond fibers, wherein a plurality of the fibers of the second layer of spunbond fibers are formed from the composition.

10. The stretchable laminate of claim 1, wherein the inorganic filler comprises an alkaline carbonate, an alkaline halide, an alkaline oxide, an alkaline sulfate, an alkali carbonate, an alkali halide, and alkali sulfate, silicon dioxide, or combinations thereof.

11. The stretchable laminate of claim 1, wherein the inorganic filler comprises calcium carbonate particles, and wherein at least a plurality of the calcium carbonate particles are coated with an organic material.

12. The stretchable laminate of claim 1, wherein the inorganic filler is present in the nonwoven substrate at a level of between 3% and 15% by weight of the nonwoven substrate

13. The stretchable laminate of claim 1, wherein the inorganic filler is present in the composition at a level of between 3% and 15% by weight of the composition.

14. The stretchable laminate of claim 1, wherein the inorganic filler in each of the formed spunbond fibers has an average particle size that is less than 90% of the diameter of the spunbond fibers, and wherein the average particle size of the inorganic filler in the formed spunbond fibers is less than 15 microns.

15. An absorbent article comprising:
a liquid pervious layer;
a liquid impervious layer;
an absorbent core disposed at least partially between the liquid pervious layer and the liquid impervious layer; and
etchable laminate of claim 1 joined to any of the liquid pervious layer, the liquid impervious layer, and the absorbent core.

16. An absorbent article comprising:
a side panel or an ear, wherein the side panel or the ear comprises the stretchable laminate of claim 1.

17. The stretchable laminate of claim 1, wherein the stretchable laminate is activated and forms a portion of a topsheet, acquisition layer, or a wing of an absorbent article.

18. A stretchable laminate for an absorbent article, the stretchable laminate comprising:
a nonwoven substrate comprising a layer of spunbond fibers, wherein a plurality of the spunbond fibers are formed from a composition comprising a thermoplastic polymer and an inorganic filler, wherein the spunbond fibers each have a diameter, and wherein the inorganic filler has an average particle size in each spunbond fiber that is less than 90% of the diameter of the spunbond fiber; and
an elastomeric material joined to the nonwoven substrate.

19. The stretchable laminate of claim 18, wherein the thermoplastic polymer comprises a polyolefin, wherein the inorganic filler comprises calcium carbonate, and wherein the average particle size of the inorganic filler is in the range of about 1 micron to about 15 microns.

20. A stretchable laminate for an absorbent article, the stretchable laminate comprising:
a first nonwoven substrate comprising:
two layers of spunbond fibers, wherein a plurality of the spunbond fibers are formed from a composition comprising a thermoplastic polymer and an inorganic filler, and wherein the inorganic filler is present in the first nonwoven substrate at a level of between 5% and 15% by weight of the first nonwoven substrate; and
a layer of meltblown fibers disposed at least partially between the two layers of spunbond fibers;
a second nonwoven substrate; and
an elastomeric material disposed at least partially between the first nonwoven substrate and the second nonwoven substrate.

21. The stretchable laminate of claim 20, wherein the second nonwoven substrate comprises a layer of carded fibers or a layer of spunbond fibers, and wherein the layer of carded fibers or the layer of spunbond fibers comprises the inorganic filler.

22. The stretchable laminate of claim 20, wherein a plurality of the meltblown fibers comprise the inorganic filler.

23. The stretchable laminate of claim 22, and wherein the inorganic filler in the plurality of the meltblown fibers has a smaller average particle size than an average particle size of the inorganic filler in the plurality of spunbond fibers.

24. The stretchable laminate of claim 20, wherein the density of the fibers of the first spunbond layer is different than the density of the fibers in the second spunbond layer.
